(19) United States
(12) Patent Application Publication
(10) Pub. No.: US 2023/0401876 A1
(43) Pub. Date: Dec. 14, 2023

(54) DATA GENERATION DEVICE, DATA GENERATION METHOD, AND RECORDING MEDIUM

(71) Applicant: Panasonic Intellectual Property Management Co., Ltd., Osaka (JP)

(72) Inventors: Masataka KUROKAWA, Osaka (JP); Yosuke MATSUSHITA, Osaka (JP); Tomoyuki NOUNO, Osaka (JP); Manabu EGAWA, Osaka (JP); Keiichi TAKAGAKI, Osaka (JP)

(73) Assignee: Panasonic Intellectual Property Management Co., Ltd., Osaka (JP)

(21) Appl. No.: 18/238,142

(22) Filed: Aug. 25, 2023

Related U.S. Application Data
(63) Continuation of application No. PCT/JP2022/002131, filed on Jan. 21, 2022.

(30) Foreign Application Priority Data
Mar. 31, 2021 (JP) 2021-059698

(51) Int. Cl. G06V 20/59; G06F 21/32

(52) U.S. Cl. CPC G06V 20/597; G06F 21/32

(57) ABSTRACT

A data generation device is a data generation device that generates determination data for determining a state of a target object. The data generation device includes: a first acquirer that acquires, from a first sensor, measurement data obtained by measuring the target object; a second acquirer that acquires, from a second sensor different from the first sensor, first factor data obtained by measuring a first noise factor that may cause noise in the measurement data; an accuracy information generator that calculates a measurement accuracy level of the measurement data based on the first factor data; and an accuracy information combiner that outputs the determination data in which the measurement data is associated with accuracy information indicating the measurement accuracy level of the measurement data.

### CROSS REFERENCE TO RELATED APPLICATIONS

This is a continuation application of PCT International Application No. PCT/JP2022/002131 filed on Jan. 21, 2022, designating the United States of America, which is based on and claims priority of Japanese Patent Application No. 2021-059698 filed on Mar. 31, 2021.

### FIELD

The present disclosure relates to a data generation device, a data generation method, and a recording medium.

### BACKGROUND

In recent years, devices that contactlessly measure target objects and acquire measurement data obtained by measuring the target objects are known. For example, Patent Literature (PTL) 1 discloses a device that acquires measurement data by contactlessly measuring a pulse wave of a target person by irradiating the target person with infrared rays and capturing an image of the target person irradiated with the infrared rays by using a camera. The measurement data acquired as described above is used to determine, for example, the state of the target person (target object), or the like.

### CITATION LIST

### Patent Literature

PTL 1: WO 2020/203914

### SUMMARY

However, the device according to PTL 1 can be improved upon. In view of this, the present disclosure provides a data generation device, a data generation method, and a recording medium that are capable of improving upon the above related art.

A data generation device according to an aspect of the present disclosure is a data generation device that generates determination data for determining a state of a target object, the data generation device including: a first acquirer that acquires, from a first sensor, measurement data obtained by measuring the target object; a second acquirer that acquires, from a second sensor different from the first sensor, first factor data obtained by measuring a first noise factor that may cause noise in the measurement data; an accuracy calculator that calculates a measurement accuracy level of the measurement data based on the first factor data; and a data outputter that outputs the determination data in which the measurement data is associated with accuracy information indicating the measurement accuracy level of the measurement data.

A data generation method according to an aspect of the present disclosure is a data generation method for generating determination data for determining a state of a target object, the data generation method including: acquiring, from a first sensor, measurement data obtained by measuring the target object; acquiring, from a second sensor different from the first sensor, first factor data obtained by measuring a first noise factor that may cause noise in the measurement data; calculating a measurement accuracy level of the measurement data based on the first factor data; and outputting the determination data in which the measurement data is associated with accuracy information indicating the measurement accuracy level of the measurement data.

A recording medium according to an aspect of the present disclosure is a non-transitory computer-readable recording medium having recorded thereon a program for causing a computer to execute the data generation method described above.

### Advantageous Effects

According to the aspects of the present disclosure, it is possible to achieve a data generation device and the like that are capable of improving upon the above related art.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1 is a diagram showing a configuration of a data generation system according to Embodiment 1.

FIG. 2 is a block diagram showing a functional configuration of an information processing device according to Embodiment 1.

FIG. 3 is a flowchart illustrating operations performed by the data generation system according to Embodiment 1.

FIG. 4 is a diagram showing data for determination and noise level according to Embodiment 1.

FIG. 5 is a diagram showing a first transformation table that shows external factor data and noise level according to Embodiment 1.

FIG. 6 is a flowchart illustrating the operation of correcting a time correlation delay shown in FIG. 3.

FIG. 7 is a diagram showing a relationship between ΔT and correlation coefficient according to Embodiment 1.

FIG. 8 is a second transformation table that shows noise level and accuracy according to Embodiment 1.

FIG. 9 is a diagram showing accuracy information-attached data according to Embodiment 1.

FIG. 10 is a flowchart illustrating the operation of determination processing shown in FIG. 3.

FIG. 11 is a diagram showing data for first determiner according to Embodiment 1.

FIG. 12 is a diagram showing data for second determiner according to Embodiment 1.

FIG. 13 is a block diagram showing a functional configuration of an information processing device according to Embodiment 2.

FIG. 14 is a flowchart illustrating operations performed by a data generation system according to Embodiment 2.

FIG. 15 is a diagram showing a third transformation table that shows first external factor data and noise level according to Embodiment 2.

FIG. 16 is a diagram showing a fourth transformation table that shows second external factor data and noise level according to Embodiment 2.

FIG. 17 is a flowchart illustrating the operation of correcting a time correlation delay shown in FIG. 14.

FIG. 18 is a diagram showing a fifth transformation table that shows noise level and accuracy according to Embodiment 2.

FIG. 19 is a diagram showing a configuration of a data generation system according to Embodiment 3.

FIG. 20 is a block diagram showing a functional configuration of an information processing device and a server device according to Embodiment 3.

FIG. 21 is a flowchart illustrating operations performed by the data generation system according to Embodiment 3.

FIG. 22 is a flowchart illustrating the operation of personal authentication shown in FIG. 21.

FIG. 23 is a flowchart illustrating operations performed by the server device according to Embodiment 3.

FIG. 24 is a flowchart illustrating the operation of calculating an external factor noise level shown in FIG. 21.

FIG. 25 is a flowchart illustrating the operation of accuracy calculation shown in FIG. 21.

FIG. 26 is a flowchart illustrating the operation of determination processing shown in FIG. 21.

FIG. 27 is a block diagram showing a functional configuration of an information processing device and a server device according to Embodiment 4.

FIG. 28 is a flowchart illustrating operations performed by the information processing device according to Embodiment 4.

FIG. 29 is a flowchart illustrating operations performed by the server device according to Embodiment 4.

FIG. 30 is a diagram showing a configuration of a data generation system according to Embodiment 5.

FIG. 31 is a flowchart illustrating operations performed by the data generation system according to Embodiment 5.

### DESCRIPTION OF EMBODIMENTS

### (Underlying Knowledge Forming Basis of the Present Disclosure)

As described in the background section above, the device disclosed in PTL 1 can be improved upon. For example, the measurement data may contain noise generated by external factors such as vibrations of the device and ambient light. If noise is contained in the measurement data, it may be difficult to accurately determine the state of the target object. To address this, studies have been conducted on techniques for removing noise from measurement data. However, it may be difficult to remove noise depending on the type of noise contained in the measurement data.

For this reason, there may be cases where priority is given to accurate determination of the state of the target object over removal of noise contained in the measurement data. However, PTL 1 does not disclose determining the state of the target object when noise is contained in the measurement data.

Accordingly, the inventors of the present application conducted in-depth studies to make further improvement for a data generation device, a data generation method, and a recording medium, and arrived at a data generation device, a data generation method, and a recording medium that can generate data based on which a state of a target object can be accurately determined even when noise is contained in measurement data.

A data generation device according to an aspect of the present disclosure is a data generation device that generates determination data for determining a state of a target object, the data generation device including: a first acquirer that acquires, from a first sensor, measurement data obtained by measuring the target object; a second acquirer that acquires, from a second sensor different from the first sensor, first factor data obtained by measuring a first noise factor that may cause noise in the measurement data; an accuracy calculator that calculates a measurement accuracy level of the measurement data based on the first factor data; and a data outputter that outputs the determination data in which the measurement data is associated with accuracy information indicating the measurement accuracy level of the measurement data.

With this configuration, the measurement accuracy level based on first factor data obtained by measuring a first noise factor that may cause noise in the measurement data is attached to the determination data. For example, by the measurement data to determine the state of the target object according to the measurement accuracy level of the measurement data, the state of the target object can be accurately determined. Accordingly, the data generation device can generate data based on which the state of the target object can be accurately determined even when noise is contained in the measurement data.

Also, for example, the measurement data and the first factor data may be time-series data obtained through measurement performed over a first period, the data generation device may further include a delay corrector that corrects a time delay between the measurement data and the first factor data, and the data outputter may output the determination data in which the measurement data whose time delay has been corrected is associated with the accuracy information.

With this configuration, the measurement data and the first factor data can be temporally associated with each other with high accuracy. For example, even when noise varies over time, the measurement data and the measurement accuracy level can be temporally associated with each other with high accuracy, and it is therefore possible to generate data based on which the state of the target object can be accurately determined even when noise is contained in the measurement data. Accordingly, the data generation device can generate determination data based on which the state of the target object can be accurately determined even when noise is contained in the measurement data.

Also, for example, the data generation device may include a third acquirer that acquires, from a third sensor different from the first sensor and the second sensor, second factor data obtained by measuring a second noise factor that may cause noise in the measurement data, and the accuracy calculator may further calculate the measurement accuracy level of the measurement data based on the second factor data.

With this configuration, the measurement accuracy level can be calculated based on two types of external factors, and it is therefore possible to more accurately calculate the measurement accuracy level. The data generation device can suppress degradation of the accuracy of calculation of the measurement accuracy level in the case where, for example, there are a plurality of noise factors that may cause noise in the measurement data.

Also, for example, the second factor data may be time-series data obtained through measurement performed over a second period at least a portion of which overlaps the first period, and the delay corrector may further correct a time delay between the measurement data and the second factor data.

With this configuration, the measurement data and the second factor data can be temporally associated with each other with high accuracy. For example, even when noise varies over time, the measurement data and the measurement accuracy level can be temporally associated with each other with high accuracy, and it is therefore possible to generate data based on which the state of the target object can be accurately determined even when noise is contained in the measurement data. Accordingly, the data generation device can generate determination data based on which the state of the target object can be more accurately determined even when noise is contained in the measurement data.

Also, for example, the data generation device may further include a noise level calculator that calculates, based on a first table in which the first factor data is associated with a noise level of noise that the first factor data adds to the measurement data, the noise level from the first factor data acquired by the second acquirer. The accuracy calculator may calculate the measurement accuracy level of the measurement data acquired by the first acquirer, from the noise level calculated by the noise level calculator, based on a second table in which the noise level is associated with the measurement accuracy level of the measurement data.

With this configuration, the measurement accuracy level can be easily calculated by performing transformation using the first table and the second table.

Also, for example, the data generation device may further include a first updater that updates at least one of the first table or the second table.

With this configuration, at least one of the first table or the second table is updated, and it is therefore possible to more accurately calculate the measurement accuracy level from noise. Also, for example, the data generation device may further include a determiner that determines the state of the target object based on the determination data output from the data outputter.

With this configuration, the data generation device can consistently perform the processing operations for determining the state of the target object.

Also, for example, the target object may be a person, and the data generation device may further include an authentication result acquirer that acquires an authentication result obtained as a result of authentication of the person.

With this configuration, determination data is generated through processing corresponding to the authentication result, and it is therefore possible to generate determination data based on which the state of the target object can be more accurately determined.

Also, for example, the data generation device may further include a second updater that updates at least one of a determination method or a threshold value that is determined based on the authentication result and to be used for the determination performed by the determiner.

With this configuration, at least one of a determination method or a threshold value that are to be used for the determination performed by the determiner is updated according to the authentication result, and it is therefore possible to determine the state of the target object corresponding to the person.

Also, for example, the determiner may include: a first determiner that determines a first state of the target object; and a second determiner that determines a second state different from the first state. The data outputter may output identical determination data to the first determiner and the second determiner, the identical determination data being the determination data for determining the state of the target object.

With this configuration, it is possible to determine a plurality of states of the target object based on one determination data.

Also, for example, the first determiner may extract, based on a first accuracy level for determining the first state, a first portion that satisfies the first accuracy level from the measurement data, and determine the first state based on the first portion extracted. The second determiner may extract, based on a second accuracy level for determining the second state, a second portion that satisfies the second accuracy level from the measurement data, and determine the second state based on the second portion extracted.

With this configuration, each of the first state and the second state can be accurately determined.

Also, for example, the second state may be a state that is less dangerous for the target object than the first state. The data generation device may further include: a first notifier that issues an emergency notification when the first determiner determines that the first state is an abnormal state; and a second notifier that outputs a sound when the second determiner determines that the second state is an abnormal state.

With this configuration, the form of notification can be changed according to the determination result, and it is therefore possible to issue a notification according to the determination result.

Also, for example, the data generation device may be mounted on a vehicle, and the target object may be a driver of the vehicle.

With this configuration, in a vehicle with too much noise to impair the accuracy of the measurement data, it is possible to generate determination data based on which the state of the target object can be accurately determined.

Also, for example, the data generation device may be mounted on a vehicle, and the target object may be a driver of the vehicle. The first determiner may determine whether the driver is having difficulty in driving the vehicle, and the second determiner may determine whether the driver is drowsy or inattentive.

With this configuration, it is possible to accurately determine whether the driver is having difficulty in driving the vehicle and also accurately determine whether the driver is drowsy or inattentive.

Also, for example, the data generation device may be mounted on a vehicle, and the target object may be a driver of the vehicle. The first determiner may determine drowsiness of the driver, and the second determiner may determine an emotion of the driver or whether the driver is ill.

With this configuration, it is possible to accurately determine the drowsiness of the driver, the emotion of the driver, or whether the driver is ill.

Also, for example, the first sensor may be a millimeter wave sensor or an infrared camera, and the second sensor may be an acceleration sensor that measures an acceleration of the vehicle.

With this configuration, it is possible generate determination data based on which the state of the target object can be accurately determined even when noise is contained in the measurement data by using acceleration that can be an external factor that may cause noise in the millimeter wave sensor or the infrared camera.

Also, for example, the data generation device may be mounted on a vehicle. The first sensor may be an infrared camera, the second sensor may be an acceleration sensor that measures an acceleration of the vehicle, and the third sensor may be a brightness sensor that measures a brightness level of surroundings of the vehicle.

With this configuration, it is possible to generate data based on which the state of the target object can be accurately determined even when noise is contained in the measurement data by using acceleration and brightness that can be external factors that may cause noise the infrared camera.

Also, for example, information for identifying the first noise factor may be acquired from the first sensor.

With this configuration, the data generation device does not need to store the information for identifying the first noise factor, and it is therefore possible to simplify the configuration of the data generation device.

Also, a data generation method according to an aspect of the present disclosure is a data generation method for generating determination data for determining a state of a target object, the data generation method including: acquiring, from a first sensor, measurement data obtained by measuring the target object; acquiring, from a second sensor different from the first sensor, first factor data obtained by measuring a first noise factor that may cause noise in the measurement data; calculating a measurement accuracy level of the measurement data based on the first factor data; and outputting the determination data in which the measurement data is associated with accuracy information indicating the measurement accuracy level of the measurement data. Also, the recording medium according to an aspect of the present disclosure is a non-transitory computer-readable recording medium having recorded thereon a program for causing a computer to execute the data generation method described above.

With this configuration, the same advantageous effects as those of the data generation device described above can be obtained.

Generic or specific aspects of the present disclosure may be implemented by a system, a method, an integrated circuit, a computer program, or a computer readable non-transitory recording medium such as a CD-ROM, or may be implemented by any combination of a system, a method, an integrated circuit, a computer program and a recording medium. The program may be stored in advance in the recording medium, or may be supplied to the recording medium via a wide area communication network such as the Internet.

Hereinafter, embodiments will be described specifically with reference to the accompanying drawings.

Embodiments described below show generic or specific examples of the present disclosure. The numerical values, shapes, structural elements, the arrangement and connection of the structural elements, steps, the order of the steps, and the like shown in the following embodiments are merely examples, and therefore are not intended to limit the scope of the present disclosure. For example, numerical values are expressions that not only have a strict meaning but also encompass a substantially equal range, for example, a margin of about several percent. Also, among the structural elements described in the following embodiments, structural elements not recited in any one of the independent claims are described as arbitrary structural elements.

In addition, the diagrams are schematic representations, and thus are not necessarily true to scale. Accordingly, for example, dimensions and the like are not necessarily the same in the diagrams. Also, in the diagrams, structural elements that are substantially the same are given the same reference numerals, and a redundant description will be omitted or simplified.

Also, in the specification of the present application, the terms that describe the relationship between elements such as “same” and the terms that describe the shape of elements such as “rectangular shape”, as well as numerical values and numerical value ranges, are expressions that not only have a strict meaning but also encompass a substantially equal range, for example, a margin of about several percent (for example, about 5%).

### Embodiment 1

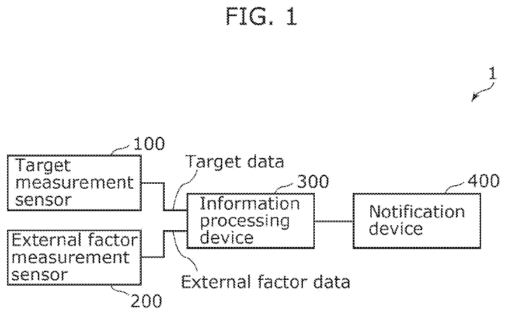

Hereinafter, a data generation system according to the present embodiment will be described with reference to FIGS. 1 to 12.

### [1-1. Configuration of Data Generation System]

First, a configuration of a data generation system according to the present embodiment will be described with reference to FIG. 1. FIG. 1 is a block diagram showing a functional configuration of data generation system 1 according to the present embodiment.

An example will be described below in which target measurement sensor 100 is mounted on a vehicle, and a target object to be measured by target measurement sensor 100 is an operator (driver) of the vehicle. An in-vehicle environment includes many factors that can generate noise that impairs the accuracy of data acquired by target measurement sensor 100, and it is therefore difficult to acquire highly accurate data even when a high accuracy sensor is mounted on the vehicle. Particularly when the frequency band of target data that needs to be acquired includes a noise frequency band, it is difficult to separate noise from the target data. In addition, ordinarily, in a vehicle, noise generated while driving the vehicle is larger than noise generated while stopping the vehicle. That is, in the vehicle, the target data that needs to be acquired may contain noise that varies over time. With this target data, it is difficult to accurately determine the state of the target object. Accordingly, data generation system 1 according to an aspect of the present disclosure is configured to generate determination data based on which a state of a target object can be determined with high accuracy even when target data contains noise that varies over time. In the description given below, the determination data may also be referred to as “accuracy information-attached data”.

Specifically, data generation system 1 is an information processing system that generates accuracy information-attached data in which data for determination based on target data obtained by target measurement sensor 100 measuring a target object is associated with a measurement accuracy level of the data for determination based on external factor data obtained by external factor measurement sensor 200 measuring an external factor of target measurement sensor 100. The target data is an example of measurement data obtained by target measurement sensor 100 measuring the target object. Hereinafter, the measurement accuracy level of the data for determination, or in other words, the measurement accuracy level of the target data may also be referred to simply as “accuracy”.

The present disclosure is not limited to a configuration in which target measurement sensor 100 is mounted on a vehicle, and the target object is not limited to a driver.

As shown in FIG. 1, data generation system 1 includes target measurement sensor 100, external factor measurement sensor 200, information processing device 300, and notification device 400. Target measurement sensor 100 is mounted on a vehicle, and measures a driver (a state of the driver) that is an example of a target object. Target measurement sensor 100 may be, for example, an on-board sensor that is mounted on a vehicle in advance (for example, mounted on a vehicle during production of the vehicle). Target measurement sensor 100 measures, for example, biometric information of the driver. For example, target measurement sensor 100 contactlessly measures the biometric information of the driver. However, the present disclosure is not limited thereto, and target measurement sensor 100 may measure the biometric information of the driver by contacting the driver. Target measurement sensor 100 is an example of a first sensor, and is implemented by, for example, a millimeter wave sensor, a camera, or the like. The camera may be, for example, an infrared camera.

Also, target measurement sensor 100 may store, for example, information for identifying a noise factor that may be contained in the target data obtained by measuring the driver. Target measurement sensor 100 may output the information to information processing device 300 when, for example, communication is established with information processing device 300. The term “information for identifying a noise factor” refers to information for identifying an external factor that may cause noise while target measurement sensor 100 is performing measurement, the external factor being, for example, vibrations of the vehicle or ambient light. The information for identifying the noise factor may be changed according to the type of target measurement sensor 100, the object on which target measurement sensor 100 is mounted, and the like.

External factor measurement sensor 200 measures an external factor that may cause noise in the target data obtained by target measurement sensor 100. It can also be said that external factor measurement sensor 200 measures an external factor that reduces the measurement accuracy level when target measurement sensor 100 measures the target object. External factor measurement sensor 200 may be, for example, an on-board sensor that is mounted on the vehicle in advance or a sensor that is retrofitted onto the vehicle after production. For example, external factor measurement sensor 200 contactlessly measures the external factor. However, the present disclosure is not limited thereto, and external factor measurement sensor 200 may measure the external factor by contacting an object that may serve as the external factor. The external factor is an example of a first noise factor.

External factor measurement sensor 200 is an example of a second sensor, and is a sensor different from target measurement sensor 100. External factor measurement sensor 200 is implemented by an acceleration sensor that measures the acceleration of the vehicle, a brightness sensor that measures the brightness of the surroundings of the vehicle, or the like. The brightness sensor may measure, for example, the brightness of the surroundings of target measurement sensor 100 (for example, the brightness of ambient light incident on target measurement sensor 100). As used herein, the term “ambient light” refers to light other than the light emitted by target measurement sensor 100.

External factor measurement sensor 200 does not measure, for example, the target object. For example, the external factor data does not include data obtained by measuring the target object. The external factor data measured by external factor measurement sensor 200 is an example of first factor data.

Information processing device 300 generates determination data for determining the state of the target object. Information processing device 300 acquires the target data from target measurement sensor 100, also acquires the external factor data from external factor measurement sensor 200, and then generate determination data based on the target data and the external factor data. Information processing device 300 generates determination data by performing processing of, for example, generating accuracy information that takes into consideration the influence of noise caused by the external factor on the target data obtained by measuring the target object, and attaching the accuracy information to the target data. Information processing device 300 is, for example, mounted on the vehicle, but may be provided in a remote location from the vehicle.

Information processing device 300 may acquire the information for identifying the noise factor from target measurement sensor 100, or store the information for identifying the noise factor. Information processing device 300 is connected to a plurality of sensors mounted on the vehicle to be capable of performing communication with each other. One of the plurality of sensors identified based on the information for identifying the noise factor may be determined as external factor measurement sensor 200.

Notification device 400 issues, to the user, a notification indicating a result of information processing performed by information processing device 300 and the like. Notification device 400 may output (present) the result of information processing and the like in the form of an image, a sound, or the like to the user. Notification device 400 includes, for example, a display device, a sound output device, and the like. The user may be a driver or a monitor who monitors (for example, remotely monitors) the driver.

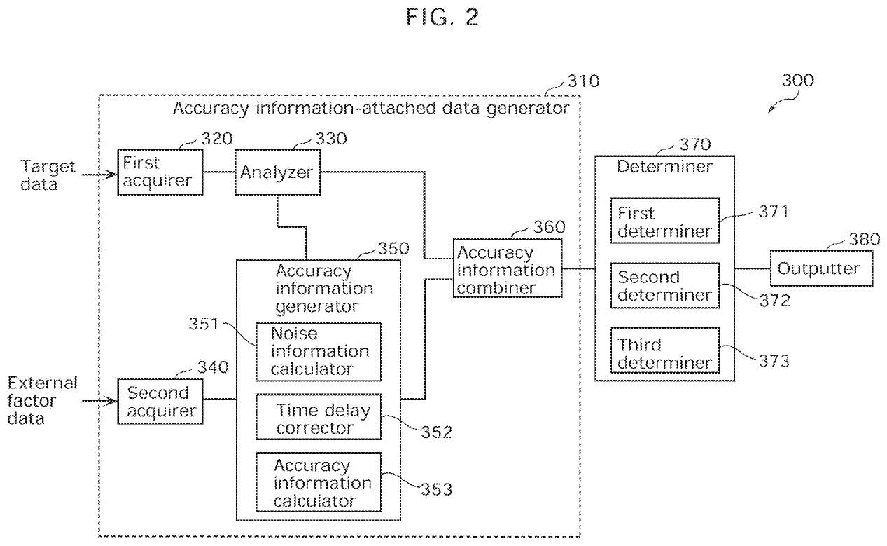

Next, a detailed configuration of information processing device 300 will be described with reference to FIG. 2. FIG. 2 is a block diagram showing a functional configuration of information processing device 300 according to the present embodiment.

As shown in FIG. 2, information processing device 300 includes accuracy information-attached data generator 310, determiner 370, and outputter 380.

Accuracy information-attached data generator 310 generates determination data based on the target data and the external factor data. Accuracy information-attached data generator 310 includes first acquirer 320, analyzer 330, second acquirer 340, accuracy information generator 350, and accuracy information combiner 360. Accuracy information-attached data generator 310 is an example of a data generation device.

First acquirer 320 acquires the target data obtained by measuring the target object from target measurement sensor 100. First acquirer 320 includes, for example, a communication interface for performing communication with target measurement sensor 100. The target data is time-series data obtained by measuring the target object over a first period, and contains time information indicating the time at which the target object was measured. The time information includes, for example, a timestamp and the like. The term “timestamp” refers to a record of the time at which data is acquired.

Analyzer 330 performs processing of transforming the target data obtained by measuring the target object into a format that can be used by determiner 370 to determine a state of the target object. Analyzer 330 transforms the target data into a feature of the target object contained in the target data. In the case where, for example, target measurement sensor 100 is a millimeter wave sensor or the like, analyzer 330 transforms the target data obtained by the millimeter wave sensor or the like performing measurement into heartbeat (heartbeat waveform) that is an example of the feature. The feature may be any one of following: the position of the head of the driver, the posture of the driver, the line of sight of the driver, the blink interval of the driver, the number of yawns of the driver, the behavior of the torso of the driver, the steering time of the driver, and the like.

Also, analyzer 330 may include a filter (not shown) for removing a predetermined component from the transformed feature. The filter is implemented by, for example, a band-pass filter or the like.

Second acquirer 340 acquires external factor data obtained by measuring the external factor that may cause noise in the target data from external factor measurement sensor 200 different from target measurement sensor 100. Second acquirer 340 includes, for example, a communication interface for performing communication with external factor measurement sensor 200. The external factor data is time-series data obtained by measuring the external factor over a first period, and contains time information indicating the time at which the external factor was measured.

Accuracy information generator 350 calculates a measurement accuracy level of the target data acquired by first acquirer 320 based on the external factor data acquired by second acquirer 340. Accuracy information generator 350 includes noise information calculator 351, time delay corrector 352, and accuracy information calculator 353. Accuracy information generator 350 is an example of an accuracy calculator.

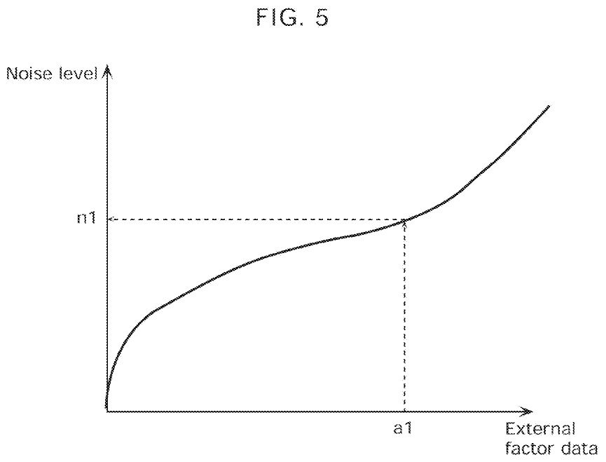

Noise information calculator 351 calculates a noise level of noise contained in the target data based on the external factor data. Noise information calculator 351 calculates the noise level from the external factor data acquired by second acquirer 340 based on, for example, a table in which external factor data is associated with a noise level of noise caused by the external factor data and applied to the target data (see, for example, a first transformation table shown in FIG. 5). The calculated noise level indicates the influence of the external factor on the target data, and is time-series data obtained through measurement performed over a first period. Noise information calculator 351 is an example of a noise level calculator. Also, the noise level is an example of noise information that indicates the degree of influence of noise on the target data. Also, the time-series data on the noise level may also be referred to as “noise level data”.

Time delay corrector 352 corrects a time delay between the target data and the external factor data. Even when the target measurement data and the external factor data are data measured during the same first period, a time difference (delay) may occur between the target measurement data and the external factor data because the sensors that measure the target measurement data and the external factor data require different times due to different data acquisition cycles, different filter processing operations performed on the data, and the like. Time delay corrector 352 corrects the time difference based on data that is based on the target data (for example, the data for determination) and data that is based on the external factor data (for example, the noise level data). Here, time delay corrector 352 is not an essential structural element. Also, time delay corrector 352 is an example of a delay corrector.

Accuracy information calculator 353 calculates the measurement accuracy level of the target data based on the external factor data acquired by second acquirer 340. Accuracy information calculator 353 calculates the measurement accuracy level of the target data acquired by first acquirer 320 based on the noise level calculated by noise information calculator 351, by using a table in which noise level and measurement accuracy level are associated with each other (see, for example, a second transformation table shown in FIG. 8). The calculated measurement accuracy is time-series data obtained through measurement performed over a first period. The information that contains measurement accuracy and time information will also be referred to as “accuracy information”. The time information used here includes, for example, a timestamp after time delay corrector 352 has corrected the time delay.

Accuracy information combiner 360 generates determination data by associating the target data with the accuracy information that contains measurement accuracy, and outputs the generated determination data. Accuracy information combiner 360 generates determination data by combining (for example, associating) the target data with the accuracy information, and outputs the generated determination data. As used herein, the term “to combine” means to generate, by using two separately acquired data items, one data item that shows a relationship between the two data items. Accuracy information combiner 360 functions as a data outputter that outputs the determination data to determiner 370. Accuracy information combiner 360 outputs the identical determination data to first determiner 371, second determiner 372, and third determiner 373.

Determiner 370 determines a state of the target object based on the determination data output from accuracy information combiner 360. Determiner 370 includes first determiner 371, second determiner 372, and third determiner 373. First determiner 371, second determiner 372, and third determiner 373 determine different states of the target object based on the determination data. Determiner 370 may determine at least one state of the target object, and include at least one of first determiner 371, second determiner 372, or third determiner 373.

First determiner 371 determines a first state of the target object. First determiner 371 extracts, based on a first accuracy level for the first state, a first portion that satisfies the first accuracy level from the target data, and determines the first state based on the extracted first portion.

First determiner 371 may determine, for example, whether the driver is having difficulty in driving the vehicle, as the first state. First determiner 371 may perform, for example, dead-man determination. The dead-man determination includes detecting a sudden change in the physical condition of the driver, a situation in which the driver is having difficulty in driving, or the like. The dead-man determination is performed by detecting a reduction in the consciousness of the driver based on at least one of the heart rate, the blink interval, or the steering time of the driver. The dead-man determination may be performed by detecting, for example, a sudden change in the heart rate of the driver. By performing the dead-man determination, it is possible to safely and automatically stop the vehicle when it is determined that the driver is having difficulty in driving the vehicle.

Second determiner 372 determines a second state different from the first state. Second determiner 372 extracts, based on a second accuracy level for the second state, a second portion that satisfies the second accuracy level from the target data, and determines the second state based on the extracted second portion.

Second determiner 372 may determine, for example, at least one of the following states: a state in which the driver is drowsy while driving the vehicle; and a state in which the driver is inattentive while driving the vehicle, as the second state. Second determiner 372 may detect, for example, the drowsiness of the driver. The drowsiness of the driver can be detected based on at least one of the heart rate, the blink interval, the steering time, the number of yawns, or image analysis of camera images (for example, the behavior of the torso of the driver).

Also, second determiner 372 may detect, for example, the tiredness of the driver. The tiredness of the driver is detected for the purpose of prompting the driver to take a rest or the like. Second determiner 372 may estimate the state of the function of the autonomic nervous system from the heartbeat, and calculate the degree of tiredness from the result of estimation.

Third determiner 373 determines a third state different from the first state and the second state. Third determiner 373 extracts, based on a third accuracy level for the third state, a third portion that satisfies the third accuracy level from the target data, and determines the third state based on the extracted third portion.

Third determiner 373 may determine, for example, at least the other one of the states described above: the state in which the driver is drowsy while driving the vehicle; and the state in which the driver is inattentive while driving the vehicle, as the third state. Third determiner 373 may detect, for example, the inattentiveness of the driver. The inattentiveness of the driver can be detected based on the line of sight of the driver or the like. The inattentiveness of the driver can be detected by, for example, monitoring the line of sight of the driver while driving the vehicle by using a camera or the like. By detecting the inattentiveness of the driver, it is possible to, when the driver is driving inattentively, warn the driver to stop the inattentive driving by using a sound or the like. Also, conditions that permit the driver to be inattentive (for example, while the vehicle is stopping) may be set. It is thereby possible to reduce erroneous determination under the above-described conditions.

The determination processing operations performed by first determiner 371, second determiner 372, and third determiner 373 are not limited to those described above. For example, first determiner 371 may determine the drowsiness of the driver, second determiner 372 may determine at least one of the emotion of the driver or whether the driver is ill, and third determiner 373 may determine the other one of the emotion of the driver or whether the driver is ill. The emotion of the driver can be used to determine whether the current driving state is a state that may cause an imminent danger, and can be detected based on, for example, the micro expression of the driver based on images. Also, whether the driver is ill is determined for the purpose of presenting, to the driver, information indicating an illness the driver is highly likely to be suffering from. A sign of facial paralysis caused by a brain stroke, eye retinopathy caused by diabetes, or the like can be determined based on images. Also, whether the driver is ill can be determined based on an electro-cardiogram, and high blood pressure, myocardial infarction, or the like can be determined.

Outputter 380 outputs the determination result obtained from determiner 370. Outputter 380 outputs the determination result to, for example, an external device through communication. Outputter 380 may be connected to, for example, notification device 400 to be capable of performing communication with each other, and may output the determination result to notification device 400.

As described above, accuracy information is attached to the determination data, and thus each of first determiner 371, second determiner 372, and third determiner 373 can extract a portion that satisfies a desired accuracy level from the determination data, and determine a state of the target object by using the extracted portion. The first accuracy level, the second accuracy level, and the third accuracy level may be stored in advance in determiner 370.

### [1-2. Operations of Data Generation System]

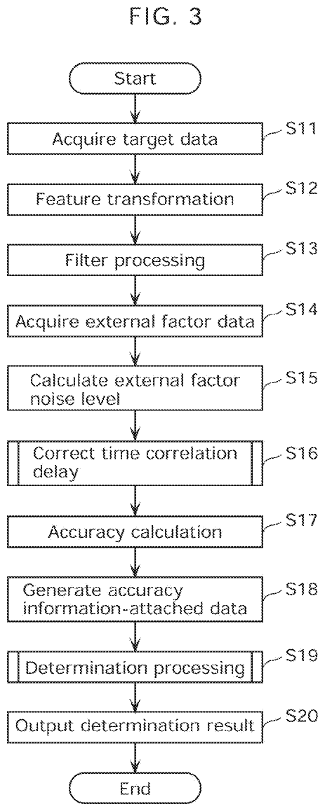

Next, operations performed by data generation system 1 configured as described above will be described with reference to FIGS. 3 to 12. FIG. 3 is a flowchart illustrating operations performed by data generation system 1 according to the present embodiment. Steps S11 to S18 shown in FIG. 3 show operations (a data generation method) performed by accuracy information-attached data generator 310 of information processing device 300, and steps S19 and S20 show operations performed by determiner 370 of information processing device 300. The data generation method includes a method for generating determination data for determining the state of the target object. It is assumed here that target measurement sensor 100 is a millimeter wave sensor, and external factor measurement sensor 200 is an acceleration sensor.

As shown in FIG. 3, first acquirer 320 acquires target data obtained by measuring the target object from target measurement sensor 100 (S11). First acquirer 320 outputs the acquired target data to analyzer 330.

Next, analyzer 330 performs feature transformation on the target data (S12). Analyzer 330 transforms the target data into a format that can be used by determiner 370. Analyzer 330 transforms the target data acquired from, for example, the millimeter wave sensor or the like into heartbeat that is an example of a feature.

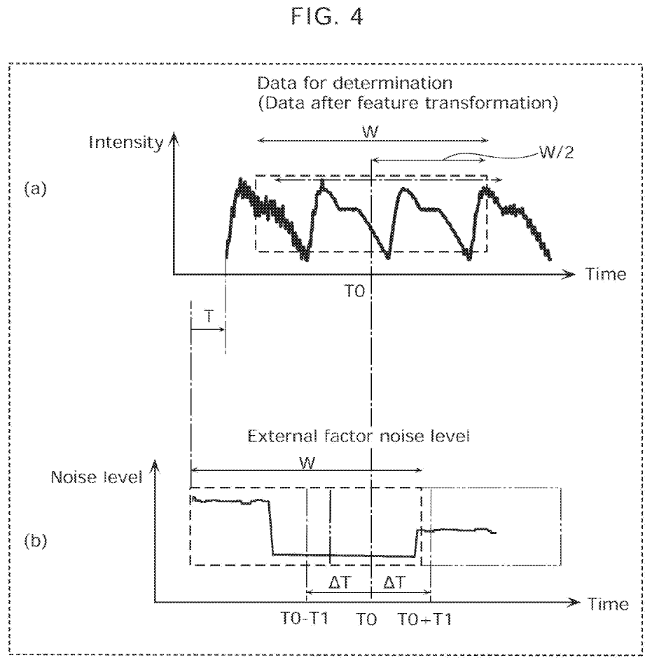

FIG. 4 is a diagram showing the data for determination and noise level according to the present embodiment.
(a) in FIG. 4 shows heartbeat data obtained by transforming the target data obtained by target measurement sensor 100 measuring the target object into heartbeat that is a feature of the target object, as an example of data for determination. As shown in FIG. 4, the data for determination contains noise whose magnitude varies with time. In (a) in FIG. 4, the horizontal axis indicates time, and the vertical axis indicates the intensity of data for determination. The time is, for example, the time identified by the timestamp, and the time at which the target data was acquired.

Referring again to FIG. 3, analyzer 33 performs filter processing on the data for determination (S13). Analyzer 330 includes, for example, a band-pass filter, and performs filter processing by using the band-pass filter. Analyzer 330 outputs the data for determination that has undergone the filter processing to accuracy information generator 350 (specifically, time delay corrector 352) and accuracy information combiner 360.

Next, second acquirer 340 acquires, from external factor measurement sensor 200, external factor data obtained by measuring the external factor (S14). Second acquirer 340 outputs the acquired external factor data to accuracy information generator 350.

Next, noise information calculator 351 calculates an external factor noise level based on the external factor data (S15). Noise information calculator 351 transforms the external factor data into noise level by using, for example, the first transformation table shown in FIG. 5.

FIG. 5 is a diagram showing a first transformation table that shows external factor data and noise level according to the present embodiment.

As shown in FIG. 5, the first transformation table is a table that shows a correspondence relationship between external factor data and noise level, with the horizontal axis indicating the intensity of the external factor data, and the vertical axis indicating the noise level. In the case where, for example, the external factor is vibration, and the external factor data is data on the acceleration of the vehicle, the horizontal axis coordinate shifts toward the right side as the acceleration is higher. For example, FIG. 5 shows an example in which, when the intensity of the external factor data is al, the noise level is n1. The noise level is indicated by, for example, a numerical value, but the present disclosure is not limited thereto.

The first transformation table is created according to the type of external factor (for example, acceleration, ambient light, or the like). The first transformation table is, for example, acquired in advance through experiments and the like, and stored in advance in a storage (not shown) included in information processing device 300. In the case where information processing device 300 stores a plurality of transformation tables, noise information calculator 351 may select, as the first transformation table, a desired table from among the plurality of transformation tables based on the type of external factor, information for identifying external factor measurement sensor 200, and the like. The storage is implemented by, for example, a semiconductor memory of the like, but the present disclosure is not limited thereto.

Noise information calculator 351 transforms the time-series external factor data into time-series noise level data (noise level data) by using the first transformation table.
(b) in FIG. 4 shows time-series external factor noise level data obtained through transformation performed by noise information calculator 351. As shown in (b) in FIG. 4, the noise level may vary with time. For example, the noise level may vary as the acceleration of the vehicle varies.

Noise information calculator 351 outputs the transformed external factor noise level to time delay corrector 352.

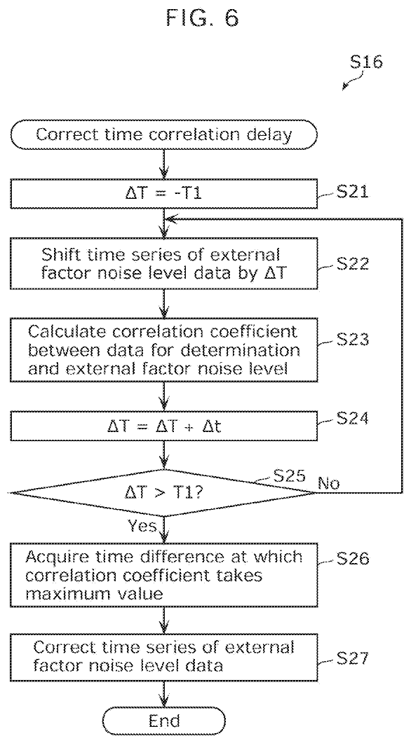

Referring again to FIG. 3, time delay corrector 352 corrects a time correlation delay between the target data and the noise level (S16). The operation of step S16 will be described with reference to FIGS. 4 and 6. FIG. 6 is a flowchart illustrating the operation (S16) of correcting the time correlation delay shown in FIG. 3.

As shown in (a) and (b) in FIG. 4, delay time T may occur between data for determination based on target data and external factor noise level based on external factor data that were obtained through measurement performed during the same period (for example, a first period). Also, it is considered that the target data obtained by measuring the target object is strongly related to the external factor data regarding the external factor that may cause noise during measurement of the target object. That is, it is considered that the data for determination and the noise level data are strongly related to each other. Accordingly, in the present embodiment, the delay (delay time T) is corrected by calculating a correlation that indicates the intensity of the relationship between the data for determination and the noise level data. For example, the delay is corrected by performing the following processing shown in FIG. 6.

As shown in FIG. 6, time delay corrector 352 sets the time difference to ΔT=−T1 (S21). As shown in (a) in FIG. 4, time delay corrector 352 places a frame (dashed frame) of time width W on the data for determination by setting time T0, which serves as a reference time point, at the center. A time width from time T0 to a frame end is indicated by W/2. Time width W may be, for example, a predetermined bit width. The frame of time width W is, for example, rectangular in shape, but the present disclosure is not limited thereto. Also, time delay corrector 352 places a frame of time width W (not shown) on the external factor noise level (noise level data) by setting time T0, which serves as a reference time point, at the center. The frame placed on the noise level data can be moved in the time axis direction. ΔT represents a time difference between time T0 and time that corresponds to the center of the frame placed on the noise level data.

Next, time delay corrector 352 shifts the time series of the external factor noise level data by an amount corresponding to ΔT (S22). Time delay corrector 352 shifts the frame placed on the noise level data in the minus direction of the time axis by T1 (see a dashed frame shown in (b) in FIG. 4).

Next, time delay corrector 352 calculates a correlation coefficient between the data for determination and the external factor noise level (S23). Time delay corrector 352 calculates the correlation coefficient between the data for determination and the external factor noise level data by, for example, convolving data within the frame (within the dashed frame) placed on the data for determination and data within the frame (within the dashed frame) when the noise-level time difference is set to ΔT=−T1.

In step S23, time delay corrector 352 clips data for a predetermined period (time width W) from the data for determination and the external factor noise level data, and calculates, based on the two clipped data items, one correlation coefficient indicating the correlation intensity.

Next, time delay corrector 352 sets the time difference to ΔT=ΔT+Δt (S24), and determines whether ΔT>T1 is satisfied (S25). In step S24, time delay corrector 352 determines whether the position of the frame shown in (b) in FIG. 4 has been moved to a position indicated by a dash-double-dotted frame, with its center being set to the position of T0+T1.

If it is determined that ΔT>T1 is not satisfied (No in S25), time delay corrector 352 returns the processing to step S22 and continues the processing. Time delay corrector 352 shifts the time width W (clipped section) of the noise level data by an amount corresponding to ΔT, which was calculated in step S24, without changing the time width W (clipped section) of the data for determination, and performs the processing operations of step S22 and subsequent steps.

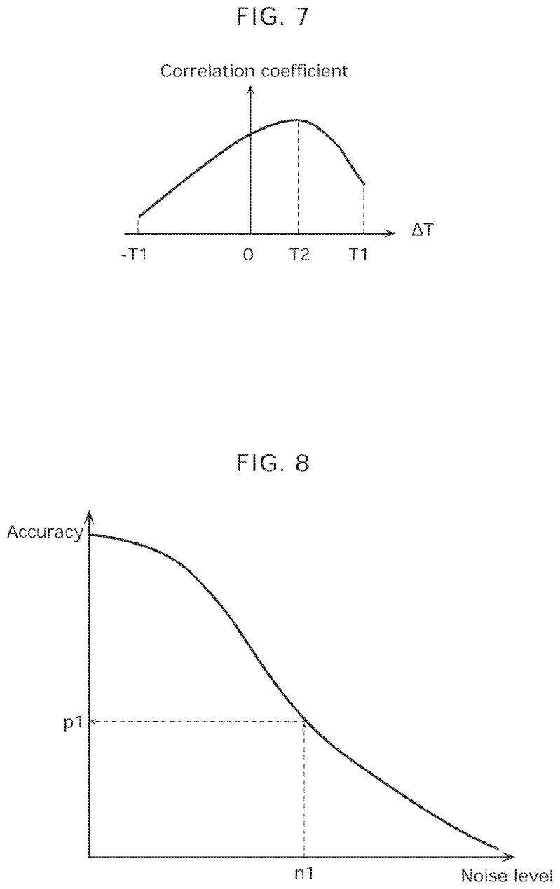

As described above, time delay corrector 352 generates a graph showing ΔT versus correlation coefficient (see FIG. 7, which will be described later) by moving the frame placed on the noise level data by an amount corresponding to Δt.

If it is determined that ΔT>T1 is satisfied (Yes in S25), time delay corrector 352 acquires a time difference at which the correlation coefficient takes a maximum value based on the graph showing ΔT versus correlation coefficient (S26). FIG. 7 is a diagram showing a relationship between ΔT and correlation coefficient according to the present embodiment.

As shown in FIG. 7, time delay corrector 352 acquires time difference T2 at which the correlation coefficient takes a maximum value. The target data and the noise level data are strongly related to each other, and it is therefore considered that the further ΔT is from the actual delay, the smaller the correlation coefficient. Accordingly, time difference ΔT (time difference T2 in the example shown in FIG. 7) at which the correlation coefficient is largest is determined as delay time T between the target data and the noise level data.

Next, time delay corrector 352 corrects the time series of the external factor noise level data based on time difference T2 acquired in step S26 (S27). Time delay corrector 352 shifts the timestamp of the noise level data by an amount corresponding to ΔT (time difference T2 in this example). It can also be said that time delay corrector 352 corrects time information that corresponds to the noise level data based on time difference T2. By doing so, the timestamp of the target data and the timestamp of the noise level data can be matched with each other.

Time delay corrector 352 outputs the noise level and the corrected timestamp (time information) to accuracy information calculator 353.

Through the processing described above, in accuracy information-attached data, which will be described later, the time information contained in the data for determination and the time information contained in the accuracy information can be matched with each other more accurately. That is, accuracy information-attached data generator 310 can generate accuracy information-attached data, with which the state of the target object can be more accurately determined.

The processing in step S16 may be omitted. For example, in the case where it is known in advance that the time difference between the target data and the noise level is within a predetermined time, the processing in step S16 may be omitted.

Referring again to FIG. 3, accuracy information calculator 353 calculates the accuracy of the target data based on the noise level data whose delay has been corrected (S17). Accuracy information calculator 353 transforms the noise level into accuracy by using, for example, a second transformation table shown in FIG. 8.

FIG. 8 is a diagram showing a second transformation table between noise level and accuracy according to the present embodiment.

As shown in FIG. 8, the second transformation table is a table that shows a correspondence relationship between noise level and the accuracy of data for determination corresponding to the noise level, with the horizontal axis indicating the noise level, and the vertical axis indicating the level of accuracy of the data for determination. For example, FIG. 8 shows an example in which the accuracy is p1 when the noise level is n1. In other words, the accuracy of the target data obtained when the external factor data is al is p1. The accuracy is indicated by, for example, a numerical value, but the present disclosure is not limited thereto.

The second transformation table is, for example, acquired in advance through experiments and the like, and stored in advance in a storage (not shown) included in information processing device 300.

Accuracy information calculator 353 transforms the time-series noise level data into time-series accuracy data by using the second transformation table. Then, accuracy information calculator 353 associates the transformed accuracy with the corrected time information, and outputs the resultant to accuracy information combiner 360.

Referring again to FIG. 3, next, accuracy information combiner 360 generates accuracy information-attached data based on the target data acquired from analyzer 330 and the accuracy information (accuracy and time information) acquired from accuracy information calculator 353 (S18). As used herein, the term “accuracy information-attached data” refers to data in which the target data and the accuracy information are associated with each other. Accuracy information combiner 360 generates accuracy information-attached data by temporally associating the target data with the accuracy based on the timestamp and the corrected time information contained in the target data.

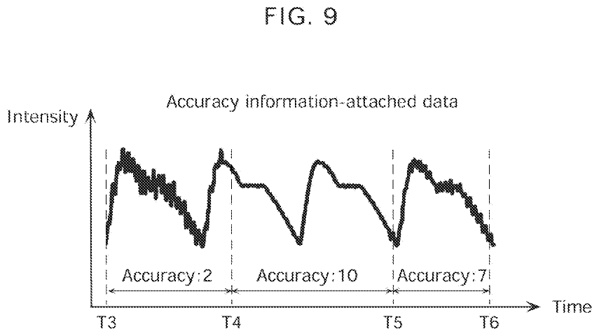

FIG. 9 is a diagram showing accuracy information-attached data according to the present embodiment. FIG. 9 shows accuracy information-attached heartbeat data as an example of accuracy information-attached data.

As shown in FIG. 9, in the accuracy information-attached data, the target data is associated with the accuracy. In the example shown in FIG. 9, the accuracy of the target data between time T3 and time T4 is 2, the accuracy of the target data between time T4 and time T5 is 10, and the accuracy of the target data between time T5 and time T6 is 7. Here, the higher the value, the higher the accuracy.

It can be seen, from this, that the accuracy of the target data between time T3 and time T4 is low, and the accuracy of the target data between time T4 and time T5 is high. As described above, accuracy information combiner 360 of the present embodiment is characterized by the feature in that information indicating the accuracy of target data is attached to the target data.

Accuracy information combiner 360 outputs the generated accuracy information-attached data to determiner 370. Specifically, accuracy information combiner 360 outputs the identical accuracy information-attached data to first determiner 371, second determiner 372, and third determiner 373. It can also be said that accuracy information combiner 360 outputs accuracy information-attached data whose time delay has been corrected and in which the target data and the accuracy information are associated with each other.

Referring again to FIG. 3, determiner 370 performs determination processing of determining the state of the target object based on the accuracy information-attached data acquired from accuracy information combiner 360 (S19). In step S19, each of first determiner 371, second determiner 372, and third determiner 373 extracts, from the accuracy information-attached data, a portion that satisfies accuracy that corresponds to the result of determination made by the determiner, and determines the state of the target object based on the extracted portion.

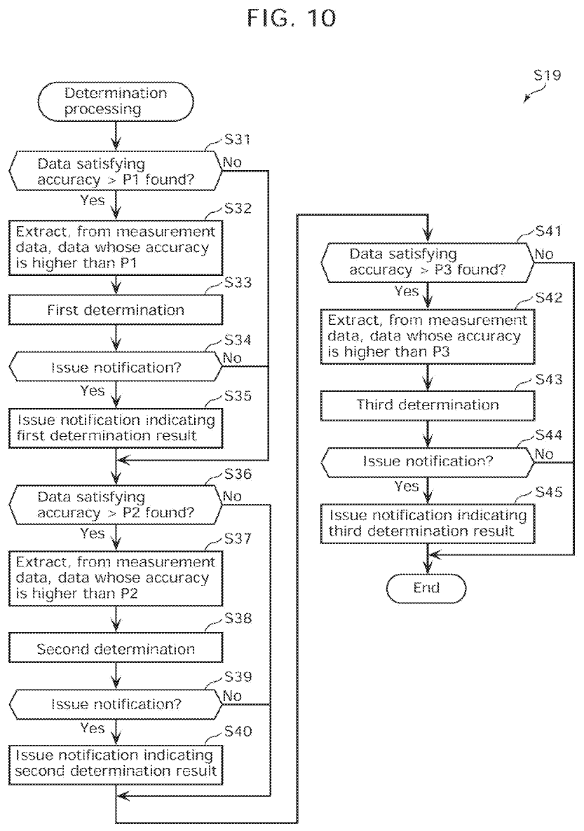

FIG. 10 is a flowchart illustrating the operation of determination processing shown in FIG. 3.

As shown in FIG. 10, first determiner 371 determines whether there is data that satisfies accuracy >P1 in the accuracy information-attached data (S31). P1 indicates an accuracy threshold value used by first determiner 371 to determine the first state of the target object (first determination), and is an example of a first accuracy level. Threshold value P1 is required accuracy that is required of the target data to accurately perform the first determination.

If it is determined that there is data that satisfies accuracy >P1 in the accuracy information-attached data (Yes in S31), first determiner 371 extracts data whose accuracy is higher than threshold value P1 from the target data (measurement data) (S32). For example, if it is determined that there is data that satisfies accuracy >P1 in the accuracy information-attached data, first determiner 371 extracts, from the accuracy information-attached data, a portion of the data for determination whose accuracy is higher than threshold value P1. The extracted data may also be referred to as “data for first determiner”. If it is determined that there is no data that satisfies accuracy >P1 in the accuracy information-attached data (No in S31), first determiner 371 proceeds to step S36 without performing the first determination.

FIG. 11 is a diagram showing the data for first determiner according to the present embodiment.

As shown in FIG. 11, in the case where threshold value P1 is 8 or more (in the case where the required accuracy is 8 or more), only data for determination between time T4 and time T5 whose accuracy is 8 or more is extracted, and the extracted data for determination is defined as data for first determiner.

Referring again to FIG. 10, first determiner 371 performs first determination based on the extracted first data for determination (S33). As described above, first determiner 371 performs first determination by extracting a portion of data for determination that satisfies a desired accuracy level from the accuracy information-attached data, and performs the first determination by using only the extracted portion. Accordingly, the first determination can be performed more accurately as compared with the case where the entire data for determination between time T3 and T6 is used.

Next, first determiner 371 determines whether it is necessary to issue a notification indicating a result of the first determination (hereinafter referred to as “first determination result”) (S34). First determiner 371 performs the determination based on, for example, a table in which first determination result is associated with information indicating whether it is necessary to issue a notification indicating the first determination result. If it is determined that it is necessary to issue a notification indicating the first determination result (Yes in S34), first determiner 371 performs processing of causing notification device 400 to issue a notification indicating the first determination result (S35). If Yes is determined in step S34, first determiner 371 outputs the first determination result to, for example, outputter 380. If it is determined that it is not necessary to issue a notification indicating the first determination result (No in S34), first determiner 371 proceeds to step S36.

Next, second determiner 372 determines whether there is data that satisfies accuracy >P2 in the accuracy information-attached data (S36). P2 indicates an accuracy threshold value used by second determiner 372 to determine the second state of the target object (second determination), and is an example of a second accuracy level. Threshold value P2 is required accuracy that is required of the target data to accurately perform the second determination.

If it is determined that there is data that satisfies accuracy >P2 in the accuracy information-attached data (Yes in S36), second determiner 372 extracts data whose accuracy is higher than threshold value P2 from the target data (S37). If it is determined that there is data that satisfies accuracy >P2 in the accuracy information-attached data, second determiner 372 extracts, from the accuracy information-attached data, a portion of data for determination whose accuracy is higher than threshold value P2. The extracted data may also be referred to as “data for second determiner”. If it is determined that there is no data that satisfies accuracy >P2 in the accuracy information-attached data, (No in S36), second determiner 372 proceeds to step S41 without performing the second determination.

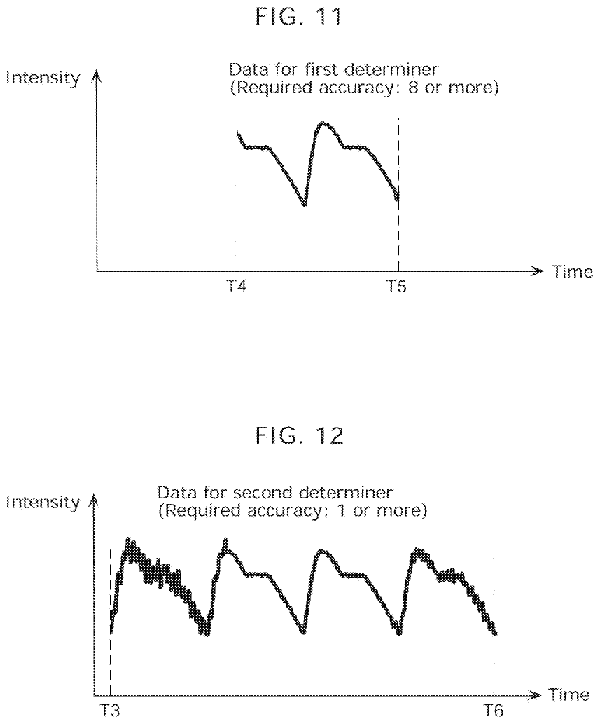

FIG. 12 is a diagram showing the data for second determiner according to the present embodiment.

As shown in FIG. 12, in the case where threshold value P2 is 1 or more (in the case where the required accuracy is 1 or more), data for determination between time T3 and time T6 whose accuracy is 1 or more is extracted, and the extracted data for determination is defined as data for second determiner. That is, the data for second determiner includes all data for determination contained in the accuracy information-attached data.

Referring again to FIG. 10, second determiner 372 performs second determination based on the extracted second data for determination (S38). As described above, threshold value P2 used to perform the second determination is low, second determiner 372 performs the second determination by using the all data for determination between time T3 and T6. Because all data for determination between time T3 and T6 is used, it is possible to suppress a situation in which the number of items of data used to perform the determination is reduced. Accordingly, second determiner 372 can suppress a situation in which accurate determination cannot be performed due to the number of items of data being reduced.

Next, second determiner 372 determines whether it is necessary to issue a notification indicating the second determination result (S39). Second determiner 372 performs the determination based on, for example, a table in which second determination result is associated with information indicating whether it is necessary to issue a notification indicating the second determination result. If it is determined that it is necessary to issue a notification indicating the second determination result (Yes in S39), second determiner 372 performs processing of causing notification device 400 to issue a notification indicating the second determination result (S40). If Yes is determined in step S39, second determiner 372 outputs the second determination result to, for example, outputter 380. If it is determined that it is not necessary to issue a notification indicating the second determination result (No in S39), second determiner 372 proceeds to step S41.

Next, third determiner 373 determines whether there is data that satisfies accuracy >P3 in the accuracy information-attached data (S41). P3 indicates an accuracy threshold value used by third determiner 373 to determine the third state of the target object (third determination), and is an example of a third accuracy level. Threshold value P3 is required accuracy that is required of the target data to accurately perform the third determination.

If it is determined that there is data that satisfies accuracy >P3 in the accuracy information-attached data (Yes in S41), third determiner 373 extracts data whose accuracy is higher than threshold value P3 from the target data (S42). If it is determined that there is data that satisfies accuracy >P3 in the accuracy information-attached data, third determiner 373 extracts, from the accuracy information-attached data, a portion of data for determination whose accuracy is higher than threshold value P3. The extracted data may also be referred to as “data for third determiner”. If it is determined that there is no data that satisfies accuracy >P3 in the accuracy information-attached data, (No in S41), third determiner 373 ends the determination processing without performing the third determination.

Third determiner 373 performs third determination based on the extracted data for third determiner (S43). As described above, third determiner 373 extracts, from the accuracy information-attached data, a portion of data for determination that satisfies the desired accuracy, and performs the third determination by using only the extracted portion. Accordingly, the third determination can be performed more accurately as compared with the case where all target data between time T3 and T6 is used.

Next, third determiner 373 determines whether it is necessary to issue a notification indicating a third determination result (S44). Third determiner 373 performs the determination based on, for example, a table in which third determination result is associated with information indicating whether it is necessary to issue a notification indicating the third determination result. If it is determined that it is necessary to issue a notification indicating the third determination result (Yes in S44), third determiner 373 performs processing of causing notification device 400 to issue a notification indicating the third determination result (S45). If Yes is determined in step S44, third determiner 373 outputs the third determination result to, for example, outputter 380. If it is determined that it is not necessary to issue a notification indicating the third determination result (No in S44), third determiner 373 ends the determination processing.

Accuracy threshold values P1, P2, and P3 may be different values. Also, threshold values P1, P2, and P3 are acquired in advance and stored in a storage (not shown) included in information processing device 300.

Various processing operations performed by first determiner 371, second determiner 372, and third determiner 373 may be performed, for example, in parallel to each other.

Referring again to FIG. 3, outputter 380 outputs the determination result acquired from determiner 370 (S20). Outputter 380 outputs the determination result to, for example, notification device 400.

Notification device 400 issues a notification indicating the acquired determination result to the user. With this configuration, only the desired determination result can be notified to the user.

### Embodiment 2

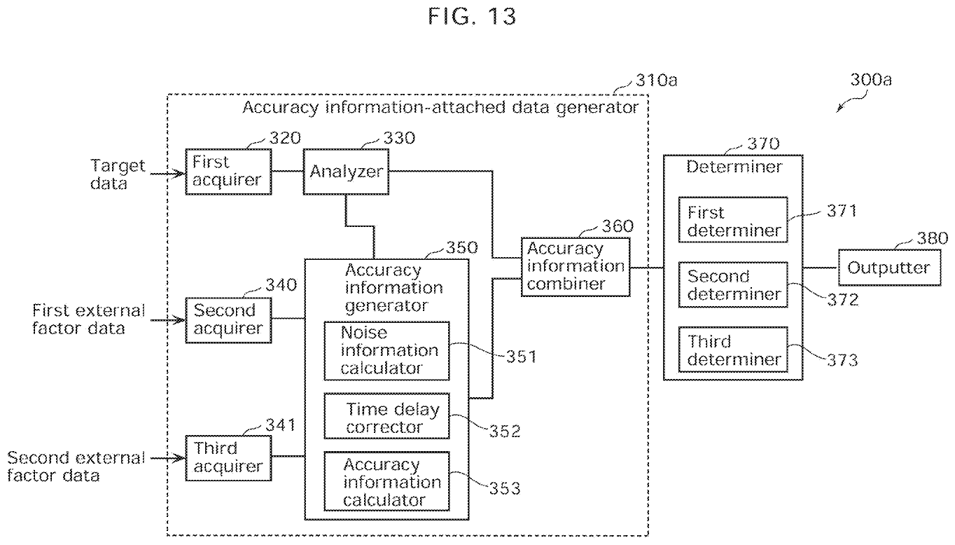

Hereinafter, a data generation system according to the present embodiment will be described with reference to FIGS. 13 to 18. The following description will be given focusing on differences from Embodiment 1, and thus a description that is the same as or similar to that of Embodiment 1 will be omitted or simplified.

### [2-1. Configuration of Data Generation System]

First, a configuration of a data generation system according to the present embodiment will be described with reference to FIG. 13. FIG. 13 is a block diagram showing a functional configuration of information processing device 300a according to the present embodiment. Information processing device 300a according to the present embodiment is different from information processing device 300 according to Embodiment 1 in that information processing device 300a includes third acquirer 341. In the present embodiment, in order to distinguish from second external factor data acquired by third acquirer 341, external factor data acquired by second acquirer 340 will be referred to as “first external factor data”. However, the data acquired by second acquirer 340 is the same as that of Embodiment 1.

In the case where target measurement sensor 100 is, for example, a camera or the like, the following i) and ii) may serve as external factors that may cause noise in the target data: i) vibrations of the vehicle; and ii) changes in ambient light. In the present embodiment, an example will be described in which accuracy information-attached data is generated by using the plurality of external factors.

As shown in FIG. 13, information processing device 300a includes accuracy information-attached data generator 310a, determiner 370, and outputter 380.

Third acquirer 341 acquires second external factor data obtained by measuring an external factor that that may cause noise in the target data from an additional external factor measurement sensor (not shown) different from target measurement sensor 100 and external factor measurement sensor 200. Third acquirer 341 includes, for example, a communication interface for performing communication with the additional external factor measurement sensor. The second external factor data is time-series data obtained by measuring the external factor over a second period at least a portion of which overlaps the first period, and contains time information that indicates time at which the external factor was measured. For example, the first period and the second period may be the same period. Also, the second external factor data is data obtained by measuring an external factor that is different from the external factor used to obtain the first external factor data.

In the present embodiment, data generation system 1 further includes an additional external factor measurement sensor other than those described above. The additional external factor measurement sensor is an example of a third sensor. The third sensor may be, for example, a brightness sensor.

Accuracy information calculator 353 calculates the measurement accuracy level based on, in addition to the first external factor data, the second external factor data. Accuracy information calculator 353 calculates one accuracy based on the first external factor data and the second external factor data.

### [2-2. Operations of Data Generation System]

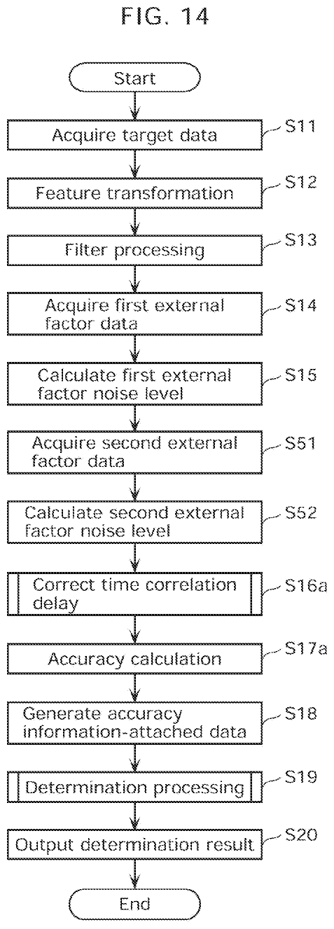

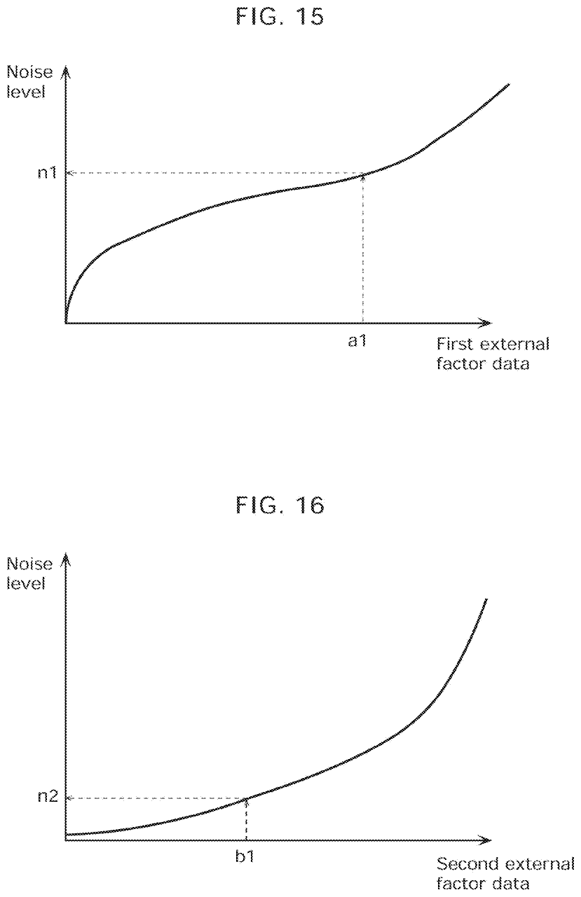

Next, operations performed by data generation system 1 configured as described above will be described with reference to FIGS. 14 to 18. FIG. 14 is a flowchart illustrating operations performed by data generation system 1 according to the present embodiment. FIG. 15 is a diagram showing a third transformation table that shows first external factor data and noise level according to the present embodiment. FIG. 16 is a fourth transformation table that shows second external factor data and noise level according to the present embodiment.

The flowchart shown in FIG. 14 includes, in addition to the steps of the flowchart shown in FIG. 3, steps S51 and S52, and also includes steps S16a and 517a in place of steps S16 and S17. Also, the third transformation table shown in FIG. 15 is the same table as the first transformation table of Embodiment 1. In step S15 shown in FIG. 14, a first external factor noise level is calculated by using the third transformation table shown in FIG. 15. However, this processing is the same as that of step S15 shown in FIG. 3, and thus a description thereof will be omitted.

As shown in FIG. 14, third acquirer 341 acquires, from the additional external factor measurement sensor, second external factor data obtained by measuring an external factor (S51). Third acquirer 341 outputs the acquired second external factor data to accuracy information generator 350.

The order in which the target data, the first external factor data, and the second external factor data are acquired is not limited to the order shown in FIG. 14.

Next, noise information calculator 351 calculates a second external factor noise level based on the second external factor data (S52). Noise information calculator 351 transforms the second external factor data into noise level by using, for example, the fourth transformation table shown in FIG. 16.

As shown in FIG. 16, the fourth transformation table is a table that shows a correspondence relationship between second external factor data and noise level, with the horizontal axis indicating the intensity of the second external factor data, and the vertical axis indicating the noise level. In the case where, for example, the second external factor is light (ambient light), and the second external factor data is data on the brightness of light incident on the vehicle, the horizontal axis coordinate shifts toward the right side as the brightness is higher. For example, FIG. 16 shows an example in which, when the intensity of the second external factor data is b1, the noise level is n2. The noise level is indicated by, for example, a numerical value, but the present disclosure is not limited thereto.

The fourth transformation table is created according to the type of second external factor (for example, acceleration, ambient light, or the like). The fourth transformation table is, for example, acquired in advance through experiments and the like, and stored in advance in a storage (not shown) included in information processing device 300a. As shown in FIGS. 15 and 16, the two transformation tables are defined by different functions.

Noise information calculator 351 transforms the second time-series external factor data into time-series noise level data by using the fourth transformation table. Then, noise information calculator 351 outputs the transformed second external factor noise level to time delay corrector 352.

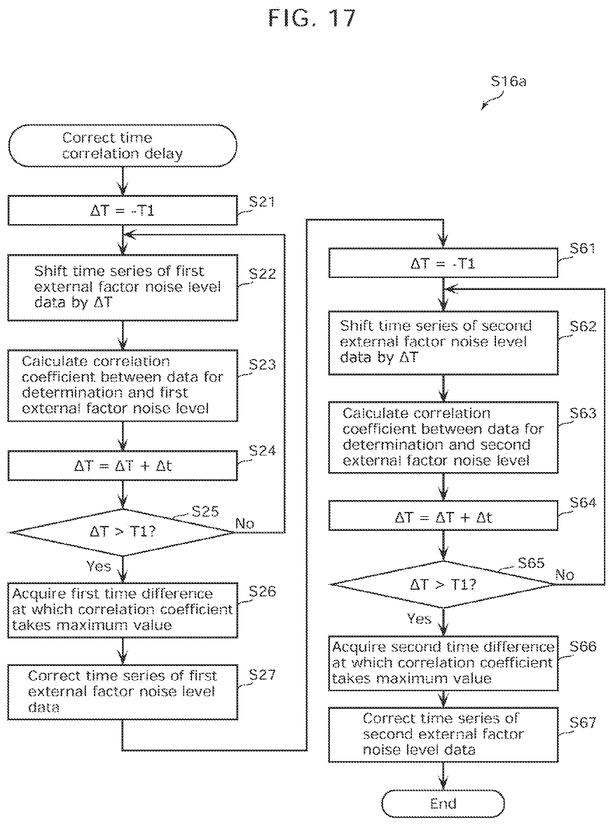

Referring again to FIG. 14, time delay corrector 352 corrects a time correlation delay between the target data and the noise level (S16a). The operation of step S16a will be described with reference to FIG. 17. FIG. 17 is a flowchart illustrating the operation (S16a) of correcting the time correlation delay shown in FIG. 14. The operations of steps S21 to S27 shown in FIG. 17 are the same as those of steps S21 to S27 shown in FIG. 6. Accordingly, a description thereof will be omitted.

As shown in FIG. 17, time delay corrector 352 of the present embodiment performs operations of steps S61 to S67 in addition to the operations of steps of Embodiment 1. Steps S61 to S67 are operations for correcting a time delay for the second external factor noise level data. It can also be said that steps S61 to S67 are operations of correcting a time correlation delay between the target data and the second external factor noise level. The operations of steps S61 to S67 are the same as those of steps S21 to S27. Accordingly, a description thereof will be omitted.

As described above, time delay corrector 352 of the present embodiment corrects, in addition to the time delay between the target data and the first external factor data, the time delay between the target data and the second external factor data.

In step S61, an example is shown in which ΔT=−T1, or in other words, ΔT in step S61 takes the same value as ΔT in step S21. However, the present disclosure is not limited thereto. ΔT in step S21 and ΔT in step S61 may take different values.

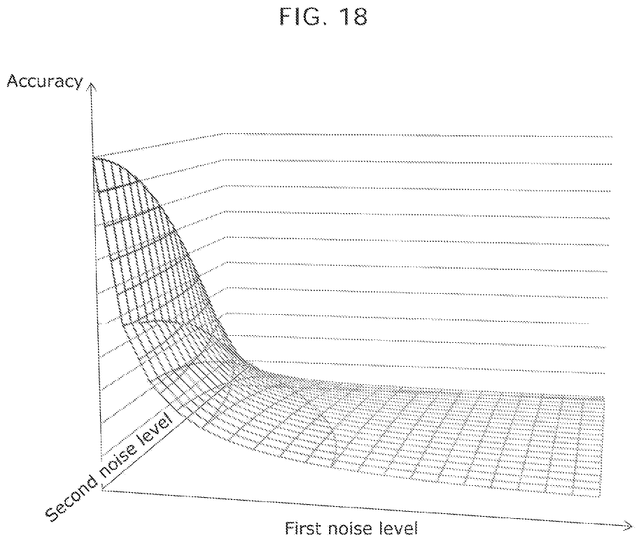

Referring again to FIG. 14, next, accuracy information calculator 353 calculates the accuracy of the target data based on the noise level data of the first external factor data whose delay has been corrected and the noise level data of the second external factor data whose delay has been corrected (S17a). Accuracy information calculator 353 transforms two noise levels into one accuracy by using, for example, a fifth transformation table shown in FIG. 18.

FIG. 18 is a diagram showing a fifth transformation table that shows noise level and accuracy according to the present embodiment.

As shown in FIG. 18, the fifth transformation table is a table that shows a correspondence relationship between the first noise level that indicates the first external factor noise level, the second noise level that indicates the second external factor noise level, and the accuracy of data for determination for the two noise levels. In FIG. 18, the table is shown in a three-dimensional Cartesian coordinate system, with the axes thereof respectively indicate the first noise level, the second noise level, and the level of accuracy of data for determination. With this configuration, one accuracy level can be acquired based on the first noise level and the second noise level.

The fifth transformation table is, for example, acquired in advance through experiments and the like, and stored in advance in a storage (not shown) included in information processing device 300a.

Accuracy information calculator 353 transforms the time-series first noise level data and the time-series second noise level data into time-series accuracy data by using the fifth transformation table. Then, accuracy information calculator 353 associates the transformed accuracy with the corrected time information, and outputs the resultant to accuracy information combiner 360.

### Embodiment 3

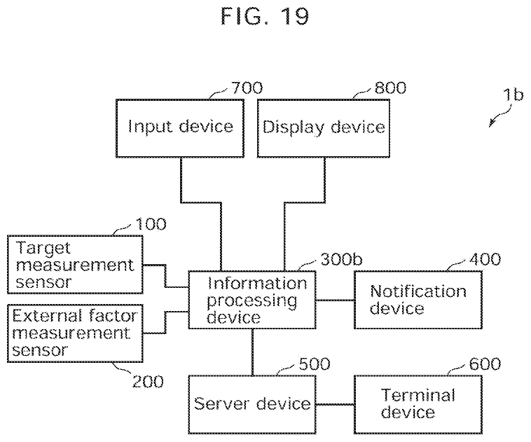

Hereinafter, a data generation system according to the present embodiment will be described with reference to FIGS. 19 to 26. The following description will be given focusing on differences from Embodiment 1, and thus a description that is the same as or similar to that of Embodiment 1 will be omitted or simplified.

### [3-1. Configuration of Data Generation System]

First, a configuration of a data generation system according to the present embodiment will be described with reference to FIG. 19. FIG. 19 is a diagram showing a configuration of data generation system 1b according to the present embodiment. Data generation system 1b according to the present embodiment is different from data generation system 1 according to Embodiment 1 in that data generation system 1b further includes server device 500, terminal device 600, input device 700, and display device 800, and also includes information processing device 300b in place of information processing device 300.

As shown in FIG. 19, data generation system 1b includes target measurement sensor 100, external factor measurement sensor 200, information processing device 300b, notification device 400, server device 500, terminal device 600, input device 700, and display device 800. Data generation system 1b updates at least one of a determination method or a threshold value (criteria for determination) used by information processing device 300b based on an authentication result obtained as a result of authentication of the target object and the like. Also, data generation system 1b further updates transformation data used by information processing device 300b. An example will be described below in which the target object is a driver, and target measurement sensor 100 and external factor measurement sensor 200 are mounted on a vehicle.

Information processing device 300b acquires, from server device 500, at least one of a determination method or a threshold value suitable for the driver based on an authentication result obtained as a result of authentication of the driver based on input information acquired by input device 700. Then, information processing device 300b performs various processing operations by using at least one of the acquired determination method or threshold value. Also, information processing device 300b performs various transformation operations by using transformation data acquired from server device 500.

Server device 500 is connected to information processing device 300b to be capable of performing communication with each other, and performs processing for outputting, to information processing device 300b, the authentication result obtained as a result of authentication of the driver and at least one of the transformation data, the determination method, or the threshold value used to perform updating.

Here, server device 500 and information processing device 300b are separate devices, but may be implemented as, for example, an integrated device.

Terminal device 600 is connected to server device 500 to be capable of performing communication with each other, and presents information acquired from server device 500. Terminal device 600 may be a device owned by the driver or a pre-set device that receives authentication results and the like transmitted. Terminal device 600 may be, for example, a portable terminal such as a smart phone or a stationary device such as a PC (personal computer).

Input device 700 receives inputs from the driver. Input device 700 is implemented by, for example, a button, a touch panel, a mouse, or the like, but may be implemented by, for example, a device that receives inputs from the driver by using sounds, gestures, or the like (for example, a microphone, a camera, or the like). Input device 700 may be configured to be capable of acquiring biometric information unique to the driver. Input device 700 may include, for example, a reader that reads fingerprints, irises of the eyes, or the like.

Display device 800 is connected to information processing device 300b to be capable of performing communication with each other, and displays, for the driver, information acquired from server device 500. Display device 800 may display, for example, the authentication result obtained by server device 500. Display device 800 is provided in, for example, an object (for example, a vehicle) on which target measurement sensor 100 and external factor measurement sensor 200 are mounted. Display device 800 is implemented by, for example, a liquid crystal display device or the like.

In the case where the object on which target measurement sensor 100 and external factor measurement sensor 200 are mounted is a vehicle, input device 700 and display device 800 may be implemented by, for example, a car navigation system.

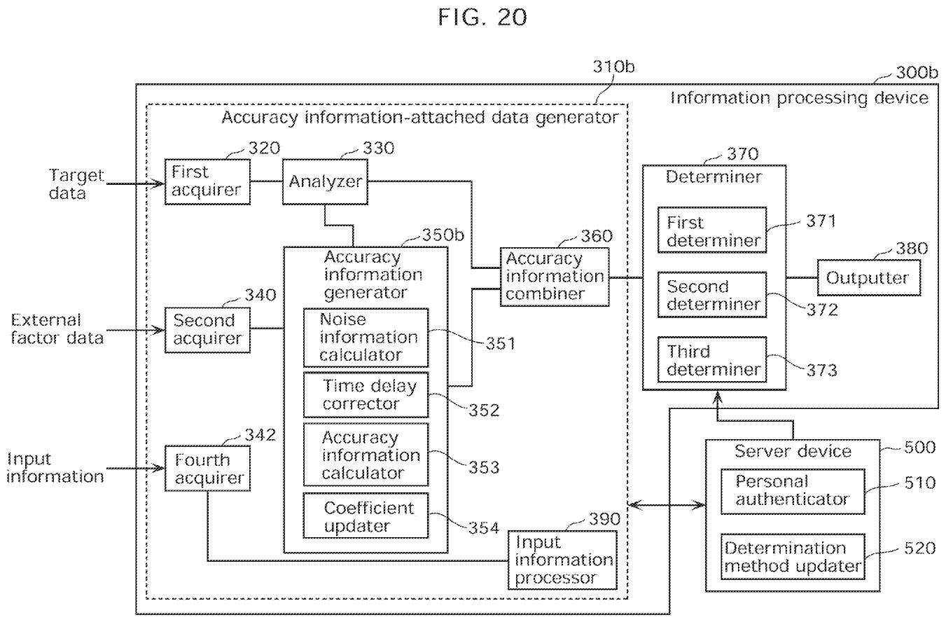

A detailed description of information processing device 300b and server device 500 will be given here with reference to FIG. 20. FIG. 20 is a block diagram showing a functional configuration of information processing device 300b and server device 500 according to the present embodiment.

As shown in FIG. 20, information processing device 300b includes, in addition to information processing device 300 according to Embodiment 1, fourth acquirer 342 and input information processor 390, and also includes accuracy information generator 350b in place of accuracy information generator 350. Information processing device 300b includes accuracy information-attached data generator 310b, determiner 370, and outputter 380.

Fourth acquirer 342 acquires, from input device 700, input information indicating an input from the driver. The input information includes information for authenticating the driver acquired from the driver. The input information contains, for example, an ID and a password (authentication information), and also contains a facial image, biometric information, and the like. However, the present disclosure is not limited thereto. Fourth acquirer 342 includes, for example, a communication interface for performing communication with input device 700. The biometric information may be information regarding, for example, fingerprint, iris, voice, or the like.

Input information processor 390 performs predetermined processing on the input information. In the present embodiment, input information processor 390 performs processing of outputting the input information acquired by fourth acquirer 342 to server device 500. In this case, input information processor 390 includes a communication interface for performing communication with server device 500. Input information processor 390 may also be connected to, for example, display device 800 to be capable of performing communication with each other.

Accuracy information generator 350b includes coefficient updater 354 in addition to accuracy information generator 350 of Embodiment 1.

Coefficient updater 354 updates, based on the information from server device 500, various types of information used in operations performed by noise information calculator 351 and accuracy information calculator 353. Coefficient updater 354 updates the first transformation table and the second transformation table. Coefficient updater 354 may, for example, access the server device that manages the first transformation table and the second transformation table, and, when the first transformation table and the second transformation table have been updated, acquire the first transformation table and the second transformation table that have been updated, and store the first transformation table and the second transformation table that were acquired in a storage (not shown). Coefficient updater 354 is an example of a first updater.

Server device 500 includes personal authenticator 510 and determination method updater 520.

Personal authenticator 510 authenticates the driver based on the input information of the driver acquired via input device 700. Personal authenticator 510 may identify the driver by, for example, comparing the input information such as the ID, the password, and the biometric information that were acquired via input device 700 with IDs, passwords, biometric information, and the like that have been registered in advance. Personal authenticator 510 may identify the driver by performing, for example, fingerprint recognition, iris recognition, facial recognition, voice recognition, or the like. The method for authenticating the driver performed by personal authenticator 510 is not limited to the method described above, and any known method can be used.

Determination method updater 520 performs processing for updating at least one of the determination method or the threshold value to at least one of a determination method or a threshold value suitable for the driver based on the authentication result obtained from personal authenticator 510.

In this example, at least input device 700 and display device 800 are mounted on, for example, an object (for example, a vehicle) on which target measurement sensor 100 and external factor measurement sensor 200 are mounted.

Input information processor 390 may perform the processing of personal authenticator 510. That is, input information processor 390 may perform processing of outputting the authentication result based on the input information. In this case, server device 500 need not include personal authenticator 510.

### [3-2. Operations of Data Generation System]

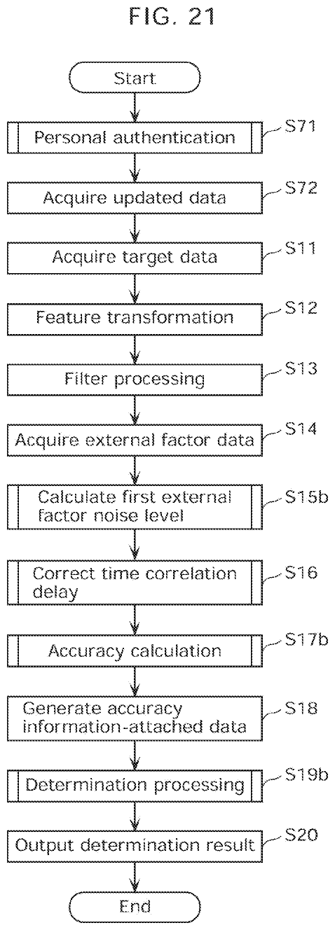

Next, operations performed by data generation system 1b configured as described above will be described with reference to FIGS. 21 to 26. FIG. 21 is a flowchart illustrating operations performed by data generation system 1b according to the present embodiment. FIG. 21 shows operations performed by information processing device 300b.

The flowchart shown in FIG. 21 includes, in addition to the steps of the flowchart shown in FIG. 3, steps S71 and S72, and also includes steps S15b, S17b, and S19b in place of steps S15, S17, and S19.

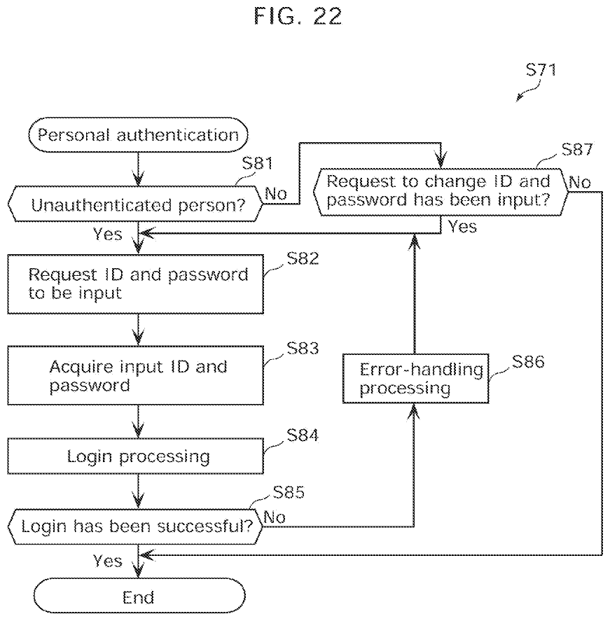

As shown in FIG. 21, input information processor 390 performs processing for personal authentication based on input information for authenticating the driver acquired by fourth acquirer 342 from the driver (S71). Input information processor 390 performs, for example, processing of outputting the input information to server device 500. FIG. 22 is a flowchart illustrating the operation (S71) of personal authentication shown in FIG. 21. FIG. 22 shows an example in which the input information includes an ID and a password.

As shown in FIG. 22, input information processor 390 determines whether the driver is an unauthenticated person (S81). Input information processor 390 acquires, for example, information such as the name of the driver via fourth acquirer 342, and determines whether the driver whose name was acquired has been authenticated. For example, input information processor 390 may perform the determination processing of step S81 every time the ignition of the vehicle is turned on or every predetermined time interval.

If it is determined that the driver is an unauthenticated person (Yes in S81), input information processor 390 requests an ID and a password to be input (S82). Input information processor 390 causes, for example, display device 800 to display a screen for inputting an ID and a password.

Next, when input information processor 390 acquires an input of an ID and a password from input device 700 via fourth acquirer 342 (S83), input information processor 390 performs login processing (S84). As the login processing, for example, input information processor 390 outputs the ID and the password to server device 500, and acquires an authentication result from server device 500. Personal authenticator 510 of server device 500 determines whether the ID and the password output from input information processor 390 match an ID and a password that have been registered in advance.

Next, input information processor 390 determines whether the login has been successful (S85). Input information processor 390 may acquire, for example, from server device 500, information indicating whether the login has been successful determined based on the determination result indicating whether the ID and the password match an ID and a password that have been registered in advance, and then determine whether the login has been successful based on the acquired information. For example, input information processor 390 may acquire the authentication result from server device 500. Input information processor 390 functions as an authentication result acquirer.

If it is determined that the login has been successful (Yes in S85), input information processor 390 ends the operation of personal authentication. If it is determined that the login has failed (No in S85), input information processor 390 performs error-handling processing (S86). As the error-handling processing, input information processor 390 may cause, for example, display device 800 to display information indicating that the login has failed (the personal authentication has failed). Then, input information processor 390 may return to step S82 and again request an ID and a password to be input.

If it is determined that the driver is an authenticated person (No in S81), input information processor 390 determines whether a change request has been input (S87). Input information processor 390 may make the determination in step S87 based on, for example, whether a request to change at least one of the ID or the password has been received from the driver.

If it is determined that a request to change at least one of the ID or the password has been input (Yes in S87), input information processor 390 proceeds to step S82, and requests a new ID and a new password to be input. If it is determined that a request to change at least one of the ID or the password has not been input (No in S87), input information processor 390 ends the operation of personal authentication.

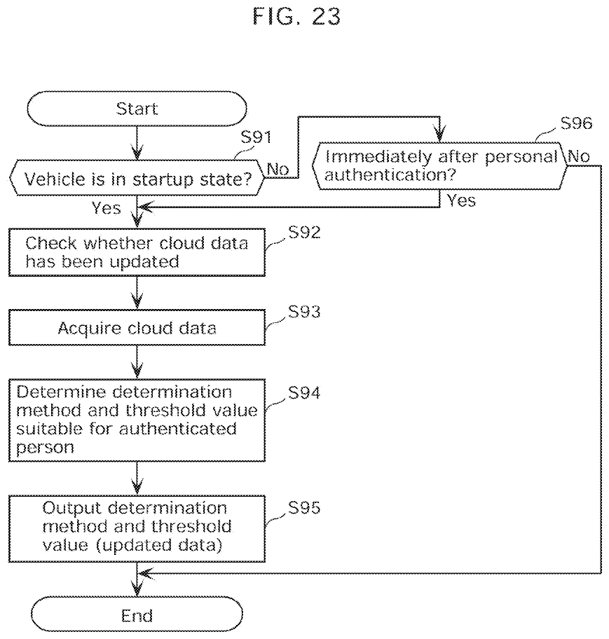

Here, operations performed by server device 500 after it is determined that the login has been successful will be described with reference to FIG. 23. FIG. 23 a flowchart illustrating operations performed by server device 500 according to the present embodiment.

As shown in FIG. 23, determination method updater 520 determines whether the vehicle is in a startup state (S91). Determination method updater 520 may determine whether the vehicle is currently in a startup state based on, for example, vehicle driving history. When an expression “the vehicle is not in a startup state” is used, it encompasses, for example, a case where a predetermined period of time or more has passed from the time of vehicle startup.

If it is determined that the vehicle is in a startup state (Yes in S91), determination method updater 520 checks whether cloud data has been updated (S92). Determination method updater 520 checks whether driver information in another server device that manages the driver information has been updated. The driver information may be, for example, information regarding medical history of the driver, an illness of the driver that needs to be reported, sleeping hours of the driver, and the like, or may be any other information regarding the driver.

Next, if it is determined that cloud data has been updated, determination method updater 520 acquires the updated cloud data (S93), and determines, based on the acquired cloud data, a determination method and a threshold value suitable for the person (driver) (S94).

In the case where, for example, first determiner 371 determines the drowsiness of the driver, second determiner 372 determines the emotion of the driver, and third determiner 373 determines the illness of the driver, determination method updater 520 may update at least one of a determination method or a threshold value for determining at least one of the drowsiness of the driver, the emotion of the driver, or the illness of the driver.

Here, an example will be described in which at least one of a determination method or a threshold value for determining the illness of the driver is updated. Determination method updater 520 acquires, as cloud data, for example, data regarding the underlying medical conditions of the driver, the chronic illnesses of the driver, and the latest diagnosis results of the driver. Also, determination method updater 520 may acquire, as cloud data, for example, information regarding illnesses the driver wants to detect while driving the vehicle that has been registered in advance by the driver.

Determination method updater 520 may determine, in step S94, a determination method and a threshold value with which it is possible to effectively detect an illness based on the illnesses included in the latest diagnosis results or the illnesses the driver wants to detect. Also, determination method updater 520 may determine, in step S94, a determination method and a threshold value with which the underlying medical conditions or chronic illnesses of the driver are not repeatedly detected.

Next, determination method updater 520 outputs the updated determination method and threshold value (updated data) to information processing device 300b (S95).

If it is determined that the vehicle is not in a startup state (No in S91), determination method updater 520 further determines whether the vehicle is in a state immediately after personal authentication (S96). If it is determined that the vehicle is in a state immediately after personal authentication (Yes in S96), determination method updater 520 proceeds to step S92. If it is determined that the vehicle is not in a state immediately after personal authentication (No in S96), determination method updater 520 ends the processing.

Determination method updater 520 may update a determination method and a threshold value for determining at least one of the drowsiness of the driver or the emotion of the driver according to the cloud data used to determine the illness of the driver.

Referring again to FIG. 21, determiner 370 acquires the updated data from server device 500 (S72). Determiner 370 may store the updated data in a storage.

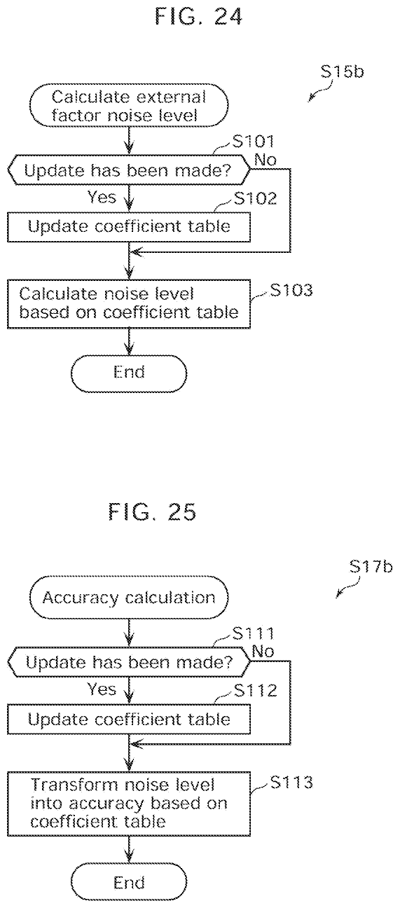

Noise information calculator 351 calculates an external factor noise level based on the external factor data (S15b). FIG. 24 is a flowchart illustrating the operation of calculating an external factor noise level shown in FIG. 21.

As shown in FIG. 24, coefficient updater 354 determines whether the first transformation table has been updated (S101). Coefficient updater 354 may, for example, access the server device that manages the first transformation table and determine whether the first transformation table has been updated. If it is determined that the first transformation table has been updated (Yes in S101), coefficient updater 354 acquires the updated first transformation table, and updates the coefficient table (for example, the first transformation table) based on the acquired first transformation table (S102). For example, coefficient updater 354 replaces the old first transformation table with the updated first transformation table.

Next, noise information calculator 351 calculates, if it is determined that the first transformation table has been updated, a noise level based on the updated coefficient table (the updated first transformation table). If it is determined that the first transformation table has not been updated (No in S101), noise information calculator 351 calculates a noise level based on the coefficient table (the first transformation table) stored in advance in a storage (S103). Step S103 is the same processing as that of step S15 shown in FIG. 3. Accordingly, a description thereof will be omitted.

Also, accuracy information calculator 353 calculates the accuracy of the target data based on the noise level data whose delay has been corrected (S17b). FIG. 25 is a flowchart illustrating the operation (17b) of accuracy calculation shown in FIG. 21.

As shown in FIG. 25, coefficient updater 354 determines whether the second transformation table has been updated (S111). Coefficient updater 354 may, for example, access the server device that manages the second transformation table, and determine whether the second transformation table has been updated. If it is determined that the second transformation table has been updated (Yes in S111), coefficient updater 354 acquires the updated second transformation table, and updates the coefficient table (for example, the second transformation table) based on the acquired second transformation table (S112). For example, coefficient updater 354 replaces the old second transformation table with the updated second transformation table.

Next, accuracy information calculator 353 transforms, if it is determined that the second transformation table has been updated, the noise level into accuracy based on the updated coefficient table (the updated second transformation table). If it is determined that the second transformation table has not been updated (No in S111), accuracy information calculator 353 transforms the noise level into accuracy based on the coefficient table (the second transformation table) stored in advance in a storage (S113). Step S113 is the same processing as that of step S17 shown in FIG. 3. Accordingly, a description thereof will be omitted.

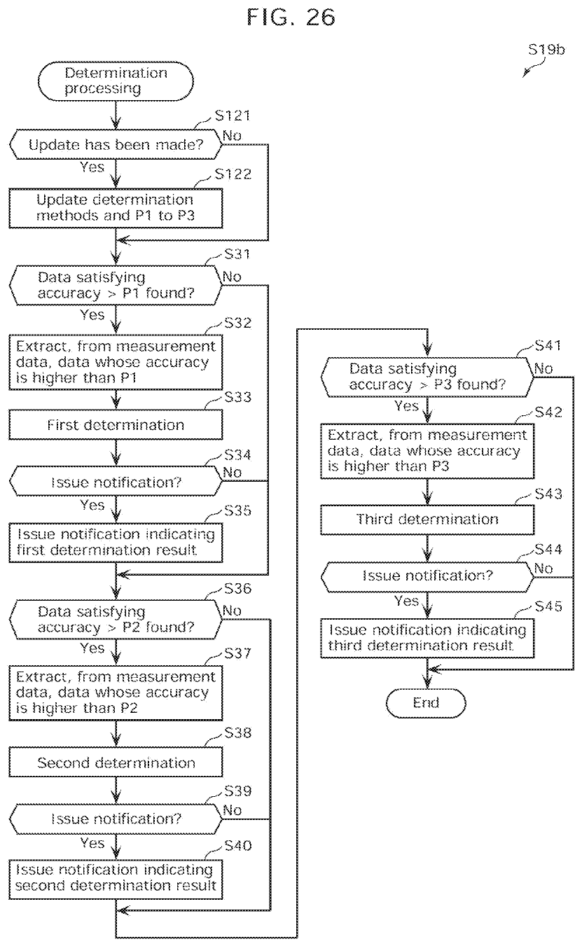

Referring again to FIG. 21, determiner 370 performs determination processing of determining the state of the target data based on the noise level data whose delay has been corrected (S19b). FIG. 26 is a flowchart illustrating the operation (19b) of determination processing shown in FIG. 21.

The flowchart shown in FIG. 26 includes, in addition to the steps of the flowchart of Embodiment 1 shown in FIG. 6, steps S121 and S122.

First, determiner 370 determines whether there has been an update to the determination methods and the threshold values (S121). Determiner 370 may perform the determination processing in step S121 based on whether an updated determination method and an updated threshold value have been acquired from determination method updater 520 after the previous determination processing.

If it is determined that there has been an update to the determination methods and the threshold values (for example, threshold values P1 to P3) (Yes in S121), determiner 370 updates the determination methods and threshold values P1 to P3 (S122). Determiner 370 replaces the determination methods and threshold values P1 to P3 used in the determination processing operations of steps S31 to S45 with the determination methods and threshold values P1 to P3 acquired from determination method updater 520. Then, determiner 370 performs the processing operations of step S31 and subsequent steps.

If it is determined that there has not been an update to the determination methods and threshold values P1 to P3 (No in S121), determiner 370 proceeds to step S31. In this case, the processing operations of step S31 and subsequent steps are performed by using the determination methods and threshold values P1 to P3 that were used in the previous determination processing.

Determiner 370 may update at least one of the determination methods or the threshold values determined based on the authentication result. Determiner 370 functions as a second updater.

### Embodiment 4

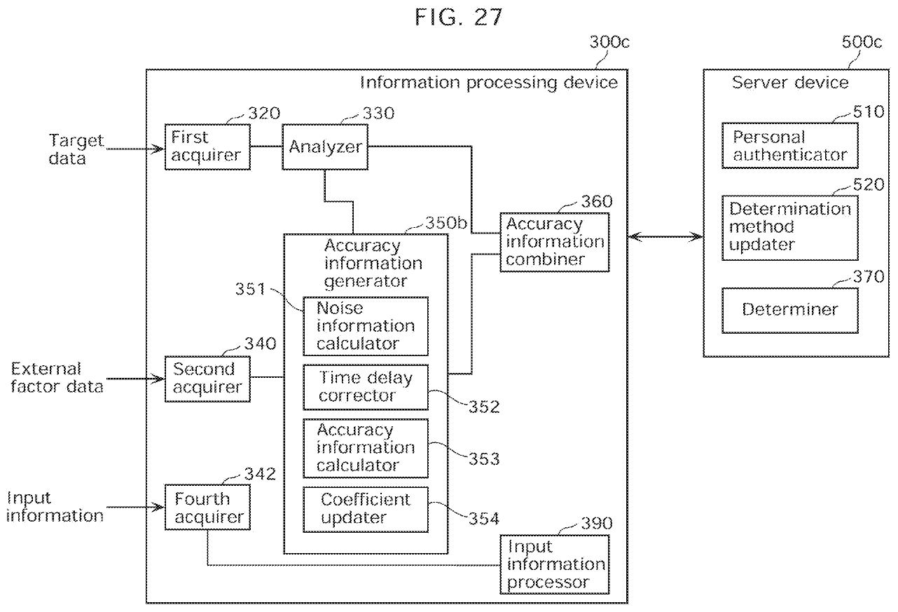

Hereinafter, a data generation system according to the present embodiment will be described with reference to FIGS. 27 to 29. The following description will be given focusing on differences from Embodiment 3, and thus a description that is the same as or similar to that of Embodiment 3 will be omitted or simplified.

### [4-1. Configuration of Data Generation System]

First, a configuration of a data generation system according to the present embodiment will be described with reference to FIG. 27. FIG. 27 is a block diagram showing a functional configuration of information processing device 300c and server device 500c according to the present embodiment. The data generation system according to the present embodiment is different from data generation system 1b according to Embodiment 3 in that server device 500 includes determiner 370.

As shown in FIG. 27, the data generation system according to the present embodiment includes information processing device 300c and server device 500c. Here, target measurement sensor 100, external factor measurement sensor 200, notification device 400, terminal device 600, input device 700, and display device 800 are the same as those of Embodiment 3, and thus an illustration thereof is omitted.

As shown in FIG. 27, information processing device 300c has the same configuration as that of information processing device 300b of Embodiment 3, except that determiner 370 and the like have been removed. Also, server device 500c has the same configuration as that of server device 500 of Embodiment 3, except that server device 500c further includes determiner 370. The configuration and the function of determiner 370 of server device 500c are the same as those of determiner 370 of Embodiment 3. Accordingly, a description thereof will be omitted.

As described above, information processing device 300c may have a configuration for generating at least accuracy information-attached data.

### [4-2. Operations of Data Generation System]

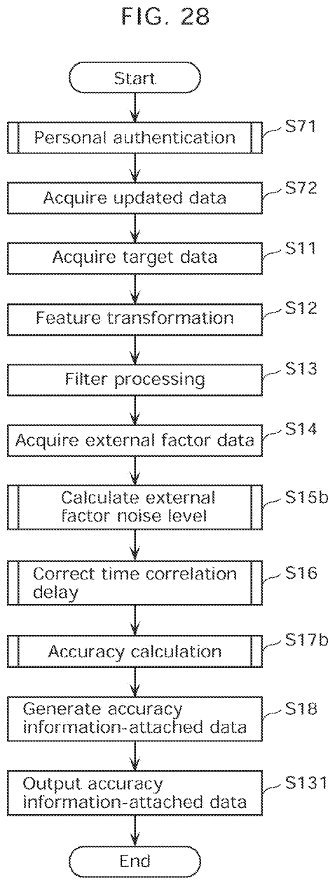

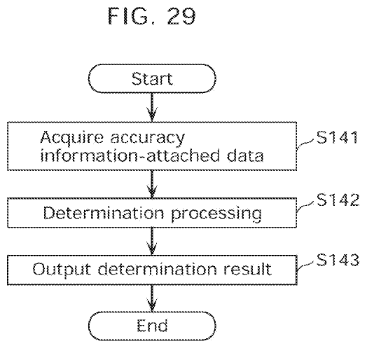

Next, operations performed by the data generation system configured as described above will be described with reference to FIGS. 28 and 29. FIG. 28 shows operations performed by information processing device 300c according to the present embodiment. The operations of steps S71 to S18 shown in FIG. 28 are the same as those of steps S71 to S18 shown in FIG. 21. Accordingly, a description thereof will be omitted. FIG. 29 is a flowchart illustrating operations performed by server device 500c according to the present embodiment. FIG. 29 shows operations performed by determiner 370 of server device 500c.

As shown in FIG. 28, when accuracy information-attached data is generated in step S18, accuracy information combiner 360 outputs the generated accuracy information-attached data to server device 500c (S131).

As shown in FIG. 29, determiner 370 of server device 500c acquires the accuracy information-attached data from information processing device 300c (S141).

Next, determiner 370 performs determination processing based on the acquired accuracy information-attached data (S142). The determination processing may be the same processing as that shown in FIG. 26

Next, determiner 370 outputs a determination result obtained as a result of the determination performed in step S142 (S143). Determiner 370 outputs the determination result to, for example, information processing device 300c. When input information processor 390 of information processing device 300c acquires the determination result (input information) from determiner 370 of server device 500c, input information processor 390 causes, for example, display device 800 to display the determination result.

Determiner 370 may output the determination result directly to display device 800. Also, determiner 370 may output the determination result to terminal device 600. Determiner 370 may further determine, for example, a proposal for health promotion for the driver based on the determination result, and output the determined proposal for health promotion to terminal device 600.

### Embodiment 5

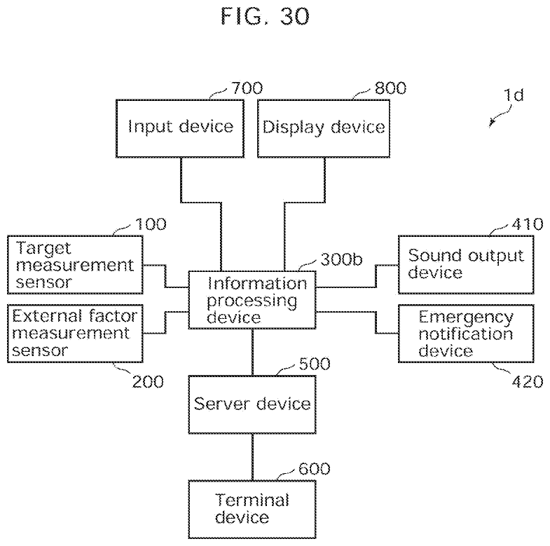

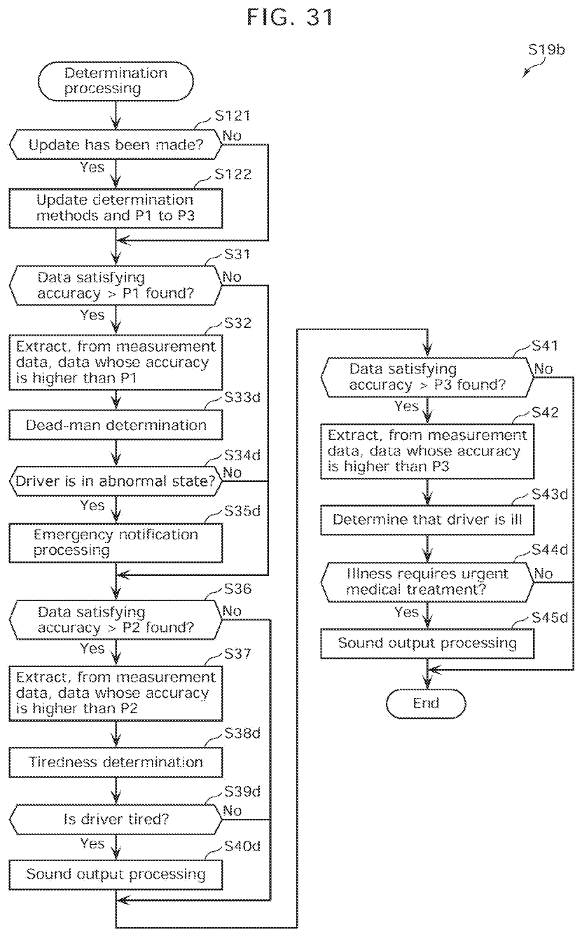

Hereinafter, a data generation system according to the present embodiment will be described with reference to FIGS. 30 and 31. The following description will be given focusing on differences from Embodiment 3, and thus a description that is the same as or similar to that of Embodiment 3 will be omitted or simplified.

### [5-1. Configuration of Data Generation System]

First, a configuration of a data generation system according to the present embodiment will be described with reference to FIG. 30. FIG. 30 is a diagram showing a configuration of data generation system 1d according to the present embodiment. Data generation system 1d according to the present embodiment is different from data generation system 1b according to Embodiment 3 in that data generation system 1d includes, as an example of notification device 400, sound output device 410 and emergency notification device 420.

As shown in FIG. 30, data generation system 1d includes target measurement sensor 100, external factor measurement sensor 200, information processing device 300b, sound output device 410, emergency notification device 420, server device 500, terminal device 600, input device 700, and display device 800. Data generation system 1d is characterized by the feature in that a different notification method for notifying a determination result is used according to the determination result obtained as a result of determination of the state of the target object by information processing device 300b.

Sound output device 410 is connected to information processing device 300b, and outputs a sound that corresponds to the determination result acquired from determiner 370 of information processing device 300b. Sound output device 410 is mounted on, for example, the vehicle, and outputs a sound that corresponds to the determination result to the driver. Sound output device 410 is implemented by, for example, a loudspeaker or the like. Data generation system 1d may include, instead of or in addition to sound output device 410, a vibration device that applies a stimulus to the driver by using vibrations, a scent generating device that applies a stimulus to the driver by using a scent, a light emitting device that that applies a stimulus to the driver by using light, and the like. Sound output device 410 is an example of a second notifier.

Emergency notification device 420 is connected to information processing device 300b, and issues an emergency notification that corresponds to the determination result acquired from determiner 370 of information processing device 300b. Emergency notification device 420 is a so-called help net device, and issues an emergency notification to, for example, an expert operator. For example, if it is determined that the driver is having difficulty in driving, emergency notification device 420 automatically transmits information indicating that the driver is having difficulty in driving to the expert operator. Emergency notification device 420 may also automatically transmit information regarding the location of the vehicle and the like to the expert operator. Emergency notification device 420 is an example of a first notifier.

Sound output device 410 and emergency notification device 420 may be included in information processing device 300b. Also, an example has been described in which data generation system 1d includes two types of notification devices, but may include three or more types of notification devices. Data generation system 1d may include different notification devices for each type of determination performed by determiner 370.

### [5-2. Operations of Data Generation System]

Next, operations performed by data generation system 1d configured as described above will be described with reference to FIG. 31. FIG. 31 is a flowchart illustrating operations performed by data generation system 1d according to the present embodiment. FIG. 31 shows operations performed in the case where first determiner 371 determines whether the driver is a dead man (dead-man determination) as first determination, second determiner 372 determines the tiredness of the driver as second determination, and third determiner 373 determines the illness of the driver as third determination. Also, a state in which whether the driver is a dead man is an example of a first state of the driver. A state in which whether the driver is tired is an example of a second state of the driver. A state in which the driver is ill is an example of a third state of the driver. For example, the second state and the third state are states that are less dangerous for the driver than the first state.

Operations that are the same or similar to those shown in FIG. 26 are given the same reference numerals as those of FIG. 26, and a description thereof will be omitted or simplified.

As shown in FIG. 31, if it is determined that there is data that satisfies accuracy >threshold value P1 in the accuracy information-attached data (Yes in S31), first determiner 371 extracts data whose accuracy is higher than threshold value P1 from the target data (measurement data) (S32), and performs dead-man determination processing of determining whether the driver is a dead man (S33d). First determiner 371 may calculate, for example, the heart rate of the driver based on the extracted portion of data for determination, and perform the determination processing in step S33d based on the calculated heart rate.

Next, if it is determined that the driver (vehicle operator) is in an abnormal state (Yes in S34d), first determiner 371 performs emergency notification processing (S35d). The emergency notification processing is processing of issuing a notification by operating, for example, at least emergency notification device 420 from among sound output device 410 and emergency notification device 420. First determiner 371 outputs, for example, information indicating that the driver is in an abnormal state to emergency notification device 420.

If it is determined by first determiner 371 that the first state is an abnormal state, emergency notification device 420 issues an emergency notification. Emergency notification device 420 may, for example, automatically transmit information indicating that the first state is an abnormal state to an expert operator.

If it is determined that the driver is not in an abnormal state (No in S34d), first determiner 371 proceeds to step S36. If Yes is determined in step S34d, the processing operations of step S36 and subsequent steps may be omitted.

Next, if it is determined that there is data that satisfies accuracy >threshold value P2 in the accuracy information-attached data (Yes in S36), second determiner 372 extracts data whose accuracy is higher than threshold value P2 from the target data (measurement data) (S37), and performs tiredness determination processing of determining whether the driver is tired (S38d). Second determiner 372 may, for example, calculate the heart rate of the driver based on the extracted portion of data for determination, and perform the determination processing in step S38d based on the calculated heart rate.

Next, if it is determined that the driver (vehicle operator) is tired (Yes in S39d), second determiner 372 performs sound output processing (S40d). The sound output processing is processing for reducing the tiredness of the driver by operating, for example, at least sound output device 410 from among sound output device 410 and emergency notification device 420. Second determiner 372 outputs, for example, information indicating that the driver is tired to sound output device 410.

For example, if it is determined by second determiner 372 that the second state is an abnormal state, sound output device 410 outputs a sound. Sound output device 410 may output, for example, a sound that prompts the driver to take a rest. Also, in the case where data generation system 1d includes a vibration device, in step S40d, vibrations may be applied to the driver to reduce the tiredness of the driver.

If it is determined that the driver is not tired (No in S39d), second determiner 372 proceeds to step S41.

Next, if it is determined that there is data that satisfies accuracy >threshold value P3 in the accuracy information-attached data (Yes in S41), third determiner 373 extracts data whose accuracy is higher than threshold value P3 from the target data (S42), and performs illness determination processing of determining whether the driver is ill (S43d). Second determiner 372 may determine, for example, whether the driver is suffering from a predetermined illness (for example, an illness that requires an urgent medical treatment such as high blood pressure or myocardial infarction) based on the extracted portion of data for determination.

Next, if it is determined that the driver (vehicle operator) is suffering from an illness that requires an urgent medical treatment (Yes in S44d), third determiner 373 performs sound output processing (S45d). The sound output processing is processing for issuing a notification indicating that the driver is suffering from an illness that requires an urgent medical treatment by operating, for example, at least sound output device 410 from among sound output device 410 and emergency notification device 420. Third determiner 373 outputs, for example, information indicating that the driver is suffering from an illness that requires an urgent medical treatment to sound output device 410.

For example, if it is determined by third determiner 373 that the third state is an abnormal state, sound output device 410 outputs a sound. Sound output device 410 may output, for example, a sound that prompts the driver to stop the vehicle or go to a hospital.

If it is determined that the driver (vehicle operator) is not suffering from an illness that requires an urgent medical treatment (No in S44d), third determiner 373 ends the determination processing.

As described above, the method for issuing a notification indicating a determination result may be selected as appropriate according to the state of the target object determined by determiner 370.

Also, as shown in FIG. 31, information processing device 300b can determine a plurality of different states of the driver by generating only one accuracy information-attached data (for example, accuracy information-attached data generated in S18 shown in FIG. 21). Information processing device 300b can, for example, determine a plurality of different states of the driver for which different data accuracy levels (for example, threshold values P1, P2, and P3) are required to determine the states from one accuracy information-attached data. For example, information processing device 300b can perform, based on one accuracy information-attached data, both determination processing that can be performed even when the data accuracy is low such as whether the driver has a heartbeat used to perform the dead-man determination and determination processing that cannot be performed unless the data accuracy is high such as whether the driver is suffering from an illness that includes a sign of heart failure. In other words, accuracy information-attached data generator 310b may generate only one accuracy information-attached data in order to determine a plurality of different states of the driver. With this configuration, it is possible to reduce, for example, the amount of throughput of accuracy information-attached data generator 310b as compared with that when a plurality of types of data are generated to determine a plurality of different states of the driver.

### Other Embodiments

Up to here, the data generation device and the like according to one or more aspects of the present disclosure have been described by way of embodiments given above, but the present disclosure is not limited to the embodiments given above. Other embodiments obtained by making various modifications that can be conceived by a person having ordinary skill in the art to any of the above embodiments as well as embodiments constructed by combining structural elements of different embodiments without departing from the scope of the present invention may also be included within the scope of the one or more aspects of the present disclosure.

For example, the target object according to the embodiments and the like given above is not limited to, for example, a driver, and may be any object. The target object may be a person, a predetermined device, or the like. The target object may be a mobile body, a stationary device, or the like. Also, the object on which at least a portion of the data generation system is mounted is not limited to a vehicle, and may be a flying object such as a drone, or an autonomous robot. Also, the object may be a mobile body or a stationary object. Examples of the vehicle include an automobile, a train, and the like.

Also, in the embodiments and the like given above, an example has been described in which the target measurement sensor and the external factor measurement sensor are contactless sensors, but the present disclosure is not limited thereto. At least one of the target measurement sensor or the external factor measurement sensor may be a contact sensor. The target measurement sensor may be, for example, a wearable terminal attached to the driver.

Also, in the embodiments and the like given above, an example has been described in which the noise level and the accuracy are indicated by numerical values, but the present disclosure is not limited thereto. The noise level and the accuracy may be indicated by a level such as “high, “medium”, or “low”.

Also, in the embodiments and the like given above, a brightness sensor is used as an example of the third sensor. However, the third sensor is not limited to a brightness sensor. In the case where the target measurement sensor is a camera, the information processing device may perform, for example, brightness transformation based on an image captured by the target measurement sensor, and acquire the intensity of ambient light (external factor intensity).

Also, in the embodiments and the like given above, an example has been described in which the first sensor and the second sensor are mounted on a vehicle, but the present disclosure is not limited thereto. The first sensor and the second sensor may be provided in, for example, a flying object such as a drone, or may be provided indoor. Alternatively, the first sensor and the second sensor may be provided in, for example, a household appliance or the like. Alternatively, the first sensor and the second sensor may be, for example, attached to a person or the like.

Also, in the embodiments and the like given above, the time delay corrector corrects the time delay of the external factor noise level data, but may correct, for example, the time delay of the external factor data. Then, the noise information calculator may transform the external factor data whose time delay has been corrected into noise level.

Also, in each of the embodiments and the like described above, the structural elements may be configured using dedicated hardware, or may be implemented by executing a software program suitable for the structural elements. The structural elements may be implemented by a program executor, such as a CPU or a processor, reading and executing a software program recorded on a recording medium such as a hard disk or a semiconductor memory.

Also, the order in which the steps of each flowchart are performed is merely an example provided to specifically describe the present disclosure. Accordingly, the order in which the steps of each flowchart are performed may be changed. Also, some of the steps may be performed simultaneously (in parallel) to other steps. Also, some of the steps need not be performed.

Also, the functional blocks shown in the block diagrams are merely examples. Accordingly, it is possible to implement a plurality of functional blocks as a single functional block, or divide a single functional block into a plurality of blocks. Alternatively, some functions may be transferred to other functional blocks. Also, the functions of a plurality of functional blocks that have similar functions may be processed by a single piece of hardware or software in parallel or by time division.

Also, the data generation system or the data generation device according to the embodiments and the like given above may be implemented by a single device or a plurality of devices. In the case where the data generation system or the data generation device is implemented by a plurality of devices, the structural elements included in the data generation system or the data generation device may be assigned to the plurality of devices in any way. In the case where the data generation system or the data generation device is implemented by a plurality of devices, the communication method for performing communication between the plurality of devices is not specifically limited, and may be wireless communication or wired communication. Also, the communication between devices may be performed by using a combination of wireless communication and wired communication.

Also, the structural elements described in the embodiments and the like given above may be implemented as software, or may typically be implemented as an LSI that is an integrated circuit. These structural elements may be implemented as individual single chips, or a part or all of these structural elements may be configured in a single chip. Here, an LSI is used, but the LSI may also be called IC, system LSI, super LSI, or ultra LSI according to the degree of integration. Also, implementation of an integrated circuit is not limited to an LSI, and may be realized by a dedicated circuit or a general-purpose processor. It is also possible to use an FPGA (Field Programmable Gate Array) that can be programmed after LSI production or a reconfigurable processor that enables reconfiguration of the connection and setting of circuit cells in the LSI. Furthermore, if a technique for implementing an integrated circuit that can replace LSIs appears by another technique resulting from the progress or derivation of semiconductor technology, the structural elements may be integrated by using that technique.

The system LSI is a super multifunctional LSI manufactured by integrating a plurality of processors on a single chip, and is specifically a computer system that includes a microprocessor, a ROM (Read Only Memory), a RAM (Random Access Memory), and the like. A computer program is stored in the ROM. The functions of the system LSI are implemented as a result of the microprocessor operating in accordance with the computer program.

Also, an aspect of the present disclosure may be a computer program that causes a computer to execute characteristic steps included in the data generation method shown in any one of FIGS. 3, 6, 10, 14, 17, 21, 22, 24 to 26, 28, and 31. Also, an aspect of the present disclosure may be a computer program that causes a computer to execute characteristic steps included in the information processing method performed by the server device shown in FIG. 23.

Also, for example, the program may be a program executed by a computer. Also, an aspect of the present disclosure may be a computer-readable non-transitory recording medium in which the program is recorded. For example, the program may be recorded in the recording medium, and then widely spread or distributed. For example, the widely spread program may be installed on a device that includes a processor. By causing the processor to execute the program, it is possible to cause the device to perform the processing operations described above.

While various embodiments have been described herein above, it is to be appreciated that various changes in form and detail may be made without departing from the spirit and scope of the present disclosure as presently or hereafter claimed.

### Further Information about Technical Background to this Application

The disclosures of the following patent applications including specification, drawings, and claims are incorporated herein by reference in their entirety: Japanese Patent Application No. 2021-059698 filed on Mar. 31, 2021, and PCT International Application No. PCT/JP2022/002131 filed on Jan. 21, 2022.

### INDUSTRIAL APPLICABILITY

The present disclosure is useful in a device or the like that generates data by measuring a target object.

1. A data generation device that generates determination data for determining a state of a target object, the data generation device comprising:
a first acquirer that acquires, from a first sensor, measurement data obtained by measuring the target object;
a second acquirer that acquires, from a second sensor different from the first sensor, first factor data obtained by measuring a first noise factor that may cause noise in the measurement data;
an accuracy calculator that calculates a measurement accuracy level of the measurement data based on the first factor data; and
a data outputter that outputs the determination data in which the measurement data is associated with accuracy information indicating the measurement accuracy level of the measurement data.

2. The data generation device according to claim 1,
wherein the measurement data and the first factor data are time-series data obtained through measurement performed over a first period,
the data generation device further comprises a delay corrector that corrects a time delay between the measurement data and the first factor data, and
the data outputter outputs the determination data in which the measurement data whose time delay has been corrected is associated with the accuracy information.

3. The data generation device according to claim 1, further comprising
a third acquirer that acquires, from a third sensor different from the first sensor and the second sensor, second factor data obtained by measuring a second noise factor that may cause noise in the measurement data,
wherein the accuracy calculator further calculates the measurement accuracy level of the measurement data based on the second factor data.

4. The data generation device according to claim 2, further comprising
a third acquirer that acquires, from a third sensor different from the first sensor and the second sensor, second factor data obtained by measuring a second noise factor that may cause noise in the measurement data,
wherein the accuracy calculator further calculates the measurement accuracy level of the measurement data based on the second factor data,
the second factor data is time-series data obtained through measurement performed over a second period at least a portion of which overlaps the first period, and
the delay corrector further corrects a time delay between the measurement data and the second factor data.

5. The data generation device according to claim 1, further comprising
a noise level calculator that calculates, based on a first table in which the first factor data is associated with a noise level of noise that the first factor data adds to the measurement data, the noise level from the first factor data acquired by the second acquirer,
wherein the accuracy calculator calculates the measurement accuracy level of the measurement data acquired by the first acquirer, from the noise level calculated by the noise level calculator, based on a second table in which the noise level is associated with the measurement accuracy level of the measurement data.

6. The data generation device according to claim 5, further comprising
a first updater that updates at least one of the first table or the second table.

7. The data generation device according to claim 1, further comprising
a determiner that determines the state of the target object based on the determination data output from the data outputter.

8. The data generation device according to claim 7,
wherein the target object is a person, and
the data generation device further comprises an authentication result acquirer that acquires an authentication result obtained as a result of authentication of the person.

9. The data generation device according to claim 8, further comprising
a second updater that updates at least one of a determination method or a threshold value that is determined based on the authentication result and to be used for the determination performed by the determiner.

10. The data generation device according to claim 7,
wherein the determiner includes:
a first determiner that determines a first state of the target object; and
a second determiner that determines a second state different from the first state, and
the data outputter outputs identical determination data to the first determiner and the second determiner, the identical determination data being the determination data for determining the state of the target object.

11. The data generation device according to claim 10,
wherein the first determiner extracts, based on a first accuracy level for determining the first state, a first portion that satisfies the first accuracy level from the measurement data, and determines the first state based on the first portion extracted, and
the second determiner extracts, based on a second accuracy level for determining the second state, a second portion that satisfies the second accuracy level from the measurement data, and determines the second state based on the second portion extracted.

12. The data generation device according to claim 10,
wherein the second state is a state that is less dangerous for the target object than the first state, and
the data generation device further comprises:
a first notifier that issues an emergency notification when the first determiner determines that the first state is an abnormal state; and
a second notifier that outputs a sound when the second determiner determines that the second state is an abnormal state.

13. The data generation device according to claim 1,
wherein the data generation device is mounted on a vehicle, and
the target object is a driver of the vehicle.

14. The data generation device according to claim 10,
wherein the data generation device is mounted on a vehicle,
the target object is a driver of the vehicle,
the first determiner determines whether the driver is having difficulty in driving the vehicle, and
the second determiner determines whether the driver is drowsy or inattentive.

15. The data generation device according to claim 10,
wherein the data generation device is mounted on a vehicle,
the target object is a driver of the vehicle,
the first determiner determines drowsiness of the driver, and
the second determiner determines an emotion of the driver or whether the driver is ill.

16. The data generation device according to claim 13,
wherein the first sensor is a millimeter wave sensor or an infrared camera, and
the second sensor is an acceleration sensor that measures an acceleration of the vehicle.

17. The data generation device according to claim 3,
wherein the data generation device is mounted on a vehicle,
the first sensor is an infrared camera,
the second sensor is an acceleration sensor that measures an acceleration of the vehicle, and
the third sensor is a brightness sensor that measures a brightness level of surroundings of the vehicle.

18. The data generation device according to claim 1,
wherein information for identifying the first noise factor is acquired from the first sensor.

19. A data generation method for generating determination data for determining a state of a target object, the data generation method comprising:
acquiring, from a first sensor, measurement data obtained by measuring the target object;
acquiring, from a second sensor different from the first sensor, first factor data obtained by measuring a first noise factor that may cause noise in the measurement data;
calculating a measurement accuracy level of the measurement data based on the first factor data; and
outputting the determination data in which the measurement data is associated with accuracy information indicating the measurement accuracy level of the measurement data.

20. A non-transitory computer-readable recording medium having recorded thereon a program for causing a computer to execute the data generation method according to claim 19.
